SEQUENCE LISTING (7 sequences)

SEQ ID NO: 1 - protein, 6 aa, Artificial Sequence
  Description of Artificial Sequence Synthetic 6x His tag
HHHHHH

SEQ ID NO: 2 - DNA, 25 bp, Artificial Sequence
  Description of Artificial Sequence Synthetic primer
aaatcacccaagcacctaagactaa

SEQ ID NO: 3 - DNA, 45 bp, Artificial Sequence
  Description of Artificial Sequence Synthetic primer
taatacgactcactatagggttgctatattaagatgtttgcgata

SEQ ID NO: 4 - DNA, 25 bp, Artificial Sequence
  Description of Artificial Sequence Synthetic primer
tacacaggtgttacaggcttaaagg

SEQ ID NO: 5 - DNA, 45 bp, Artificial Sequence
  Description of Artificial Sequence Synthetic primer
taatacgactcactatagggaagccctagtaaggtatcaagcatt

SEQ ID NO: 6 - DNA, 20 bp, Artificial Sequence
  Description of Artificial Sequence Synthetic primer
ttgtgagcgaagctatggtg

SEQ ID NO: 7 - DNA, 20 bp, Artificial Sequence
  Description of Artificial Sequence Synthetic primer
cagcttcttgccaagctttt

(19) United States
(12) Patent Application Publication
(10) Pub. No.: US 2009/0053259 A1
(43) Pub. Date: Feb. 26, 2009

(54) VACCINES AND METHODS FOR TREATMENT OR PREVENTION OF GRAM NEGATIVE BACTERIAL INFECTION IN A VERTEBRATE SUBJECT

(75) Inventors: Andrew A. Potter, Saskatoon (CA); Ursla Fernando, Saskatoon (CA)

Correspondence Address: FENWICK & WEST LLP, SILICON VALLEY CENTER, 801 CALIFORNIA STREET, MOUNTAIN VIEW, CA, 94041, US

(73) Assignee: University of Saskatchewan, Saskatoon (CA)

(21) Appl. No.: 12/178,436

(22) Filed: Jul. 23, 2008

Related U.S. Application Data
(60) Provisional application No. 60/961,769, filed on Jul. 23, 2007.

(51) Int. Cl. A61K 39/106

(52) U.S. Cl. 424/190.1; 424/234.1

(57) ABSTRACT

Methods for the treatment or prevention of Gram negative bacterial infection in a vertebrate subject are provided. The methods provide administering an antagonist of bacterial flagellar protein biosynthesis to the vertebrate subject in an amount effective to reduce or eliminate the bacterial infection. Methods for the treatment or prevention of Campylobacter jejuni infection in an avian species or a mammalian species are provided.

### CROSS REFERENCE TO RELATED APPLICATION

This application claims the benefit of U.S. provisional patent application Ser. No. 60/961,769, filed Jul. 23, 2007 which is incorporated by reference in its entirety.

### FIELD

The invention relates to vaccines and methods for the treatment or prevention of Gram negative bacterial infection in a vertebrate subject. Methods are provided for administering a bacterial flagellar protein or a protein involved in bacterial flagellar protein biosynthesis to the vertebrate subject in an amount effective to reduce or eliminate the bacterial infection. Methods for the treatment or prevention of Campylobacter jejuni infection in an avian species or a mammalian species are provided.

### BACKGROUND

Campylobacter jejuni is the leading bacterial cause of food-borne gastroenteritis in developed countries. Although C. jejuni can be found in most warm blooded animals, its most favoured habitat appears to be the intestine of avian species in which up to 109 colony forming units per gram of feces can be found (Newell and Feamley, Appl Environ Microbiol 69:4343-4351, 2003). Ingestion of as few as 100 cells is sufficient to colonize the avian gut, and it has been reported that in broiler houses a total flock is colonized within a few days from initial exposure (Young et al., Avian Dis 43:763-767, 1999). In the intestine, C. jejuni is thought to penetrate the mucus that covers the epithelium of the small intestine and caeca. Important for this colonization event is the characteristic corkscrew-like motility conferred by the polar flagellum and the spiral shape of C. jejuni. Flagella and motility have also been implicated in adhesion and invasion by C. jejuni (Wassenaar et al., EMBO J. 10:2055-2061, 1991) and many studies have shown the importance of flagella for virulence of C. jejuni (Grant et al., Infect Immun 61:1764-1771, 1993; Yao et al., 1993; Lee et al., 1986), although the specific aspect of flagellar function that contributes to virulence remains unclear.

Based on the genome sequence of C. jejuni NCTC11168, more than 40 genes are predicted to be involved in the assembly and function of the flagella. In Gram-negative bacteria, flagellar biosynthesis is regulated in a hierarchical cascade, with genes expressed in the order in which they are required for the assembly of the flagellar structure. The initiation of transcription by sigma factors is a key step in this process. Of the three sigma factors (σ⁷⁰, σ⁵⁴ and σ²⁸) identified in the C. jejuni genome sequence,

σ⁵⁴ and σ²⁸ are responsible for regulation of a number of flagellar biosynthesis and structural genes (Jagannathan et al., J Bacteriol 183:2937-2942, 2001; Hendrixson et al, Mol Microbiol 40:214-224, 2001). Carrillo et al (2004) demonstrated that two variants of C. jejuni NCTC11168 with markedly different virulence properties, including colonization in poultry, invasion of Caco-2 cells and motility, the genes which showed higher expression in the colonizing strain (V1) relative to the poorly colonizing strain (V26) belonged to the late flagellar genes, and many of the genes with differences in expression levels between the two variants were regulated by either σ²⁸ or σ⁵⁴ promoter.

C. jejuni strains appear to use the flagellar structure as a type III secretory organelle in the absence of a specialized secretion system and secretes Cia proteins upon co-culture with either host cells or serum-supplemented tissue culture medium (Konkel et al, Mol Microbiol 32:691-701, 1999; Rivera-Amill, V. & Konkel, M. E. (1999) Secretion of Campylobacter jejuni Cia proteins is contact dependent. In Mechanisms in the pathogenesis of enteric diseases, pp 225-229. Edited by P S Paul and D H Francis, New York: Plenum Publishing Corp.). These secreted Cia proteins are required for the maximal invasion of host epithelial cells by C. jejuni (Konkel et al., Mol Microbiol 32:691-701, 1999). Mutation of the ciaB gene resulted in a loss of secretion of all Cia proteins and the loss of the invasive phenotype (Konkel et al., Mol Microbiol 32:691-701, 1999). Furthermore, Konkel et al (2004) showed that the C. jejuni Cia proteins are secreted from the flagellar export apparatus and secretion of these proteins requires a functional basal body, hook, and at least one of the filament proteins.

In the flagellar morphogenesis pathway, the flagellar assembly process begins with the basal body, followed by the hook and, finally, the filament. In Salmonella species it has been shown that upon the completion of hook assembly, FlgD, the hook capping protein, is displaced from the hook and FlgK, the hook filament junction protein is assembled at the hook tip (Kazumasa et al., 1999). They showed that the flgk mutants of Salmonella cannot assemble a filament, but that the hook length is similar to the wild type levels. In Campylobacter, the role of the hook filament junction protein FlgK, is unknown, except it has been shown that flgk mutants are defective in their motility phenotype (Golden and Acheson, Infect Immun 70:1761-1771, 2002). Since colonization of the intestinal tract has been shown to be multifactorial, in this study we tested the in vitro adhesion, invasion and secretion competencies of the fliA, rpoN and flgk mutants of C. jejuni strain NCTC11168 and their ability to colonize the chicken in vivo.

A need exists in the art for improved vaccines and methods for treatment or prevention of Gram negative bacterial infection, for example, C. jejuni infection, of a vertebrate subject such as avian species or mammalian species.

### SUMMARY

The present invention generally relates to vaccines and methods for the prevention or treatment of Gram negative bacterial infection in a vertebrate subject. Methods for the treatment or prevention of Campylobacter jejuni infection in an avian species or a mammalian species are provided. The methods provide administering a bacterial flagellar protein or a protein involved in bacterial flagellar protein biosynthesis to the vertebrate subject in an amount effective to reduce or eliminate the Gram negative bacterial infection. A vaccine composition is provided comprising an effective immunizing amount of a bacterial flagellar protein or a protein involved in bacterial flagellar biosynthesis and a pharmaceutically acceptable carrier, wherein said vaccine composition is effective in vertebrates against Gram-negative bacterial infection. The Gram negative bacterial infection can be a Camylobacter jejuni infection. In particular, the bacterial flagellar protein can be bacterial FlgK protein. The protein involved in bacterial flagellar protein biosynthesis can be RpoN protein or FliA protein. The protein involved in bacterial flagellar protein biosynthesis can be a bacterial protein that is regulated by RpoN protein or a bacterial protein that is regulated by FliA protein. The protein involved in bacterial flagellar protein biosynthesis can be a biosynthetic intermediate of a bacterial flagellar protein.

The vaccine composition can be administered to avian species or mammalian species. For example, the vaccine composition can be administered to an avian species to prevent transfer of the infectious C. jejuni bacteria from avian species to mammalian species and to reduce the risk of infection to mammals, e.g., humans. The avian subject can be one day of age or older or can be in ovo. The vaccine can further comprise an immunological adjuvant, for example, an oil-in-water emulsion, or a mineral oil and dimethyldioctadecylammonium bromide. The immunological adjuvant can be VSA3. The VSA3 can be present in the composition at a concentration of about 20% to about 40% (v/v), or at a concentration of about 30% (v/v). The vaccine composition can further comprise one or more recombinant or purified antigens selected from the group consisting of FlgK protein, RpoN protein, or FliA protein. The vaccine composition can further comprise one or more recombinant or purified antigens selected from the group consisting of a bacterial protein regulated by RpoN protein, or a bacterial protein regulated by FliA protein. One or more of FlgK protein, RpoN protein, or FliA protein can comprise at least 20% of the cell protein present in the composition.

A method for preventing or treating a Gram-negative bacterial infection in a vertebrate subject is provided which comprises administering a bacterial flagellar protein or a protein involved in bacterial flagellar biosynthesis to the vertebrate subject in an amount effective to reduce or eliminate the bacterial infection. The Gram negative bacterial infection can be a Camylobacter jejuni infection. In particular, the bacterial flagellar protein can be bacterial FlgK protein. The protein involved in bacterial flagellar protein biosynthesis can be RpoN protein or FliA protein. The protein involved in bacterial flagellar protein biosynthesis can be a bacterial protein that is regulated by RpoN protein or a bacterial protein that is regulated by FliA protein. The protein involved in bacterial flagellar protein biosynthesis can be a biosynthetic intermediate of a bacterial flagellar protein.

The method further provides administering the bacterial flagellar protein or a protein involved in bacterial flagellar biosynthesis to avian species or mammalian species. For example, the vaccine composition can be administered to an avian species to prevent transfer of the infectious C. jejuni bacteria from avian species to mammalian species and to reduce the risk of infection to mammals, e.g., humans. The avian subject can be one day of age or older or can be in ovo. The vaccine can further comprise an immunological adjuvant, for example, an oil-in-water emulsion, or a mineral oil and dimethyldioctadecylammonium bromide. The immunological adjuvant can be VSA3. The VSA3 can be present in the composition at a concentration of about 20% to about 40% (v/v), or at a concentration of about 30% (v/v). The vaccine composition can further comprise one or more recombinant or purified antigens selected from the group consisting of FlgK protein, RpoN protein, or FliA protein. The vaccine composition can further comprise one or more recombinant or purified antigens selected from the group consisting of a bacterial protein regulated by RpoN protein, or a bacterial protein regulated by FliA protein. One or more of FlgK protein, RpoN protein, or FliA protein can comprise at least 20% of the cell protein present in the composition.

A method for eliciting an immunological response in a vertebrate subject against a Gram-negative bacterial infection is provided which comprises administering a bacterial flagellar protein or a protein involved in bacterial flagellar biosynthesis to the vertebrate subject in an amount effective to reduce or eliminate the bacterial infection. The Gram negative bacterial infection can be a Camylobacter jejuni infection. In particular, the bacterial flagellar protein can be bacterial FlgK protein. The protein involved in bacterial flagellar protein biosynthesis can be RpoN protein or FliA protein. The protein involved in bacterial flagellar protein biosynthesis can be a bacterial protein that is regulated by RpoN protein or a bacterial protein that is regulated by FliA protein. The protein involved in bacterial flagellar protein biosynthesis can be a biosynthetic intermediate of a bacterial flagellar protein.

A method for reducing colonization of Gram negative bacteria in an avian species is provided which comprises administering to the avian species a composition comprising a bacterial flagellar protein or a protein involved in bacterial flagellar biosynthesis and an immunological adjuvant in an amount effective to reduce Gram negative bacterial count in the avian species. The method for reducing colonization of Gram negative bacteria in the avian species further comprises reducing a risk of infectious transfer from the avian species to humans.

The bacterial flagellar protein can be a bacterial FlgK protein. The protein involved in bacterial flagellar biosynthesis can be a bacterial RpoN protein or a bacterial FliA protein. The protein involved in bacterial flagellar biosynthesis can be regulated by a bacterial RpoN protein. The protein involved in bacterial flagellar biosynthesis can be regulated by a bacterial FliA protein.

### BRIEF DESCRIPTION OF THE DRAWINGS

FIG. 1 shows PCR confirmation of insertional inactivation of the rpoN and flgk mutants of C. jejuni NCTC11168.

FIG. 2 shows motility of the C. jejuni wild-type (CJ1) and mutants after incubation for 48 hr at 37° C. on 0.4% MH agar.

FIG. 3 shows electron micrographs of wild-type C. jejuni (CJ1) and the mutant strains.

FIG. 4 shows secretion of Cia proteins by C. jejuni wild type (CJ1), fliA, rpoN and flgk mutants.

FIG. 5 shows adherence to and invasion of Hela cells with wild-type C. jejuni (CJ1), fliA, rpoN and flgk mutants.

FIG. 6 shows caecal colonization levels (cfu/gm) of wild-type C. jejuni (CJ1), fliA, rpoN and flgk mutants.

### DETAILED DESCRIPTION

The present invention generally relates to vaccines and methods for the prevention or treatment of Gram negative bacterial infection in a vertebrate subject. Methods for the treatment or prevention of Campylobacter jejuni infection in an avian species or a mammalian species are provided. The methods provide administering a bacterial flagellar protein or a protein involved in bacterial flagellar protein biosynthesis to the vertebrate subject in an amount effective to reduce or eliminate the Gram negative bacterial infection. In particular, the bacterial flagellar protein can be bacterial FlgK protein. The protein involved in bacterial flagellar protein biosynthesis can be RpoN protein or FliA protein. The protein involved in bacterial flagellar protein biosynthesis can be a bacterial protein regulated by RpoN protein or a bacterial protein regulated by FliA protein. The protein involved in bacterial flagellar protein biosynthesis can be a biosynthetic intermediate of a bacterial flagellar protein.

Vaccine compositions and method are provided for reducing colonization of Gram negative bacteria in an avian species comprising administering to the avian species a composition comprising a bacterial flagellar protein or a protein involved in bacterial flagellar biosynthesis and an immunological adjuvant in an amount effective to reduce Gram negative bacterial count in the avian species. The method further comprises reducing a risk of infectious transfer from the avian species to humans.

Aspects of the present invention demonstrates a role for flagellar sigma factor σ²⁸ (FliA protein) and the alternative sigma factor σ⁵⁴ (RpoN protein) of Campylobacter jejuni NCTC11168 in colonization of chickens. Campylobacter jejuni, a common commensal Gram-negative motile bacterium commonly found in chickens is a frequent cause of human gastrointestinal infections. The polar flagellum of C. jejuni is an important virulence and colonization factor, providing motility to the cell as well a type III secretion function. The flagellar biosynthesis genes fliA (σ²⁸) and rpoN (σ⁵⁴) of C. jejuni regulate a large number of genes involved in motility, protein secretion and invasion, which have been shown to be important factors for the virulence of this organism. To understand the role of the flagellar sigma factors, σ²⁸ and σ⁵⁴, in regulating colonization of the chicken intestinal tract, we assessed fliA and rpoN mutants of C. jejuni NCTC11168 for their ability to secrete Cia proteins and to adhere to and invade cultured cells of human and chicken origin. The mutants were also tested for their in vivo colonization potential in a chicken model with two different challenge doses. The fliA mutant was 75% less motile than the wild type but secreted Cia proteins, yet it did not colonize the chicken cecum. The rpoN mutant cells lacked the spiral shape of C. jejuni and motility was reduced by 90%. The rpoN mutant did not secrete any Cia proteins but RT-PCR analysis showed the presence of ciaB mRNA, indicating that ciaB gene expression was independent of σ⁵⁴. Not surprisingly, the colonization defects of both fliA and rpoN mutants were more severe than flagellar structural mutants, indicating the presence of mechanisms regulating the virulence-associated genes of C. jejuni that are independent of σ²⁸ and σ⁵⁴. The present invention further demonstrated that FlgK, the hook filament junction protein of C. jejuni, is required for assembly of the flagellar secretory apparatus and a flgk mutant of C. jejuni expressing only the hook is non motile and completely attenuated for caecal colonization in chickens.

It is to be understood that this invention is not limited to particular methods, reagents, compounds, compositions or biological systems, which can, of course, vary. It is also to be understood that the terminology used herein is for the purpose of describing particular aspects only, and is not intended to be limiting. As used in this specification and the appended claims, the singular forms “a”, “an” and “the” include plural references unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Vertebrate,” “mammal,” “subject,” or “patient” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, avian species, ducks, geese, chickens, amphibians, and reptiles.

“Avian” and “avian subjects” refers to males and females of any avian species, but is primarily intended to encompass poultry which are commercially raised for eggs, meat or as pets. Accordingly, the terms “avian” and “avian subject” are particularly intended to encompass chickens, turkeys, ducks, geese, quail, pheasant, parakeets, parrots, and the like. The avian subject can be a hatched bird, which term encompasses newly-hatched (i.e., about the first three days after hatch) as well as post-hatched birds such as, for example, adolescent, and adult birds. The avian subject can also be pre-hatch, i.e., in ovo.

“Treating” or “treatment” refers to either (i) the prevention of infection or reinfection, e.g., prophylaxis, or (ii) the reduction or elimination of symptoms of the disease of interest, e.g., therapy. “Treating” or “treatment” can refer to the administration of a vaccine composition comprising a bacterial flagellar protein or a protein antigen involved in bacterial flagellar protein biosynthesis. Treating an avian or mammalian species with the vaccine composition can prevent or reduce the risk of infection to humans. Treatment can be prophylactic (to prevent or delay the onset of the disease, or to prevent the manifestation of clinical or subclinical symptoms thereof) or therapeutic suppression or alleviation of symptoms after the manifestation of the disease. “Treating” or “treatment” can also refer to the administration of an antibody compositions, compounds or agents, peptide, peptidomimetic, a small chemical inhibitor RNA, short hairpin RNA, ribozyme, or antisense oligonucleotide of the present invention to prevent or delay the onset of the symptoms, complications, or biochemical indicia of a disease resulting from Gram negative bacterial infection, alleviating the symptoms or arresting or inhibiting further development of the disease, condition, or disorder (e.g., Gram negative bacterial infection).

“Preventing” or “prevention” refers to prophylactic administration or vaccination with antigen formations, for example, an antigen comprising a bacterial flagellar protein or a protein involved in bacterial flagellar protein biosynthesis. Preventing infection with a Gram negative bacteria, e.g., C. jejuni, refers to preventing colonization of an avian or mammalian species. Morbidity or mortality can result from infection or colonization of an avian species or mammalian species. “Preventing” or “prevention” can also refer to antibody compositions, compounds or agents, peptide, peptidomimetic, a small chemical inhibitor RNA, short hairpin RNA, ribozyme, or antisense oligonucleotide of the present invention prior to exposure of the vertebrate subject to the Gram negative bacteria to prevent or significantly reduce the level of Gram negative bacterial infection in the vertebrate subject.

“Therapeutically-effective amount” or “an amount effective to reduce or eliminate bacterial infection” refers to an amount of an antagonist of bacterial flagellar protein synthesis that is sufficient to prevent Gram-negative bacterial infection or to alleviate (e.g., mitigate, decrease, reduce) at least one of the symptoms associated with Gram negative bacterial infection. It is not necessary that the administration of the compound eliminate the symptoms of Gram negative bacterial infection, as long as the benefits of administration of compound outweigh the detriments. Likewise, the terms “treat” and “treating” in reference to Gram negative bacterial infection, as used herein, are not intended to mean that the avian subject is necessarily cured of infection or that all clinical signs thereof are eliminated, only that some alleviation or improvement in the condition of the avian subject is effected by administration of the an antagonist of bacterial flagellar protein synthesis.

“FlgK” or FlgK protein” refers to a gene or polypeptide encoding a hook filament junction protein involved in bacterial flagellar protein biosynthesis.

“RpoN” or RpoN protein” refers to a gene or polypeptide encoding σ⁵⁴, alternative sigma factor, involved in bacterial flagellar protein biosynthesis.

“FliA” or FliA protein” refers to a gene or polypeptide encoding σ²⁸, flagellar sigma factor, involved in bacterial flagellar protein biosynthesis. The protein products of middle class operons include proteins necessary for the structure and assembly of the hook-basal body, an intermediate structure in flagellar assembly, and the transcriptional regulators FlgM and σ²⁸. The class 3 promoters are specific for σ²⁸ RNA polymerase.

“A protein involved in bacterial flagellar biosynthesis” refers to a protein such as RpoN protein or FliA protein that is involved in the synthesis of flagellar protein components.

“A protein involved in bacterial flagellar biosynthesis that is regulated by a bacterial RpoN protein” refers to a protein that is a regulatory target of the RpoN protein. Virulence genes are regulated by σ⁵⁴ (σ^{N}) in multiple bacterial species.

“A protein involved in bacterial flagellar biosynthesis that is regulated by a bacterial FliA protein” refers to a protein that is a regulatory target of the FliA protein.

σ²⁸ (FliA) protein and σ⁵⁴ (RpoN protein) regulate a large number of genes involved in motility, protein secretion and invasion, which have been shown to be important factors for the virulence of C. jejuni.

Flagellar transcriptional hierarchy is coupled to flagellar assembly. The synthesis and function of the flagellar and chemotaxis system require the expression of more than 50 genes which are divided among at least 17 operons that constitute the large, coordinately regulated flagellar regulon. These genes are transcribed in operons of three temporal classes, early, middle, and late. In general, late genes are downregulated in strains defective in early or middle genes, and neither middle nor late genes are expressed in strains defective in early class genes. The three promoter classes that correspond to the transcriptional classification have differences in DNA sequence. The class 1 promoter is a single promoter that transcribes the two early genes in the flhDC operon. The flhD and flhC genes encode transcriptional activators, and transcription of the flhDC operon responds to many environmental cues. The FlhD and FlhC proteins are transcriptional activators for the class 2 promoters upstream of the middle gene operons. The protein products of middle class operons include proteins necessary for the structure and assembly of the hook-basal body, an intermediate structure in flagellar assembly, and the transcriptional regulators FlgM and σ²⁸. The class 3 promoters are specific for σ²⁸ RNA polymerase. With the exception of the hook-associated proteins (HAPs), gene products required at the late assembly stage are transcribed only from class 3 promoters.

The transcriptional classes appeared to correspond to major steps in morphological development of the flagellar structure. However, this relatively simple transcriptional hierarchy was complicated by the fact that many of the genes are expressed from more than one promoter class. For example, the flgK, flgL, flgM, flgN, fliD, fliS, and fliT genes are transcribed from both FlhDC-dependent class 2 promoters and 6²⁸-dependent class 3 promoters. The coupling of gene expression to flagellar assembly is accomplished by the regulation of σ²⁸ activity by the anti-σ²⁸ factor FlgM. Regulation of FlgM levels in response to flagellar assembly results in the temporal regulation of σ²⁸-dependent transcription to ensure the efficiency of assembly. In addition to complex transcriptional control, there are layers of posttranscriptional regulation, which are also believed to ensure an efficient temporal assembly process.

The early genes are included in the master flagellar operon, flhDC. The FlhC and FlhD proteins form a heteromultimeric complex that directs σ⁷⁰-dependent transcription of class 2 promoters of the middle and some late genes. The middle operons encode structural and assembly proteins required for the biosynthesis of the flagellar motor intermediate structure, also known as the hook-basal body. In addition to hook-basal body proteins, two competing regulatory proteins, FlgM and FliA (σ²⁸), are also transcribed from class 2 promoters. The fliA gene encodes an alternative transcription factor, σ²⁸, which is specifically required for class 3 promoter transcription. Genes whose products are required late in flagellar assembly, including the external filament (flagellar propeller), are primarily transcribed from class 3 promoters. The FlgM protein binds to σ²⁸ directly to prevent class 3 promoter transcription until after hook-basal body completion. Once the hook-basal body is complete, FlgM is secreted from the cell to free s28, and class 3 transcription occurs. In this way, the external filament (propeller) is not made until there is a motor (hook-basal body) for it to polymerize onto. A number of late flagellar genes, including flgK, flgL, flgM, flgN, fliD, fliS, and fliT, are expressed in both middle and late operons. Chilcott and Hughes, Microbiology and Molecular Biology Reviews 64: 694-708, 2000.

σ⁵⁴ forms a distinct subfamily of sigma factors, apart from the σ⁷⁰-like family. In almost all species, the σ⁵⁴ factor is called σ^{N}. σ^{N} has been identified in many species, spanning a diverse phylogeny, including Legionella pneumophila, Pseudomonas spp., Enterococcus faecalis, Campylobacter jejuni, and Listeria monocytogenes. A physiological theme for σ^{N}-dependent genes is provided wherein the regulated genes described to date control a wide diversity of processes. Often nitrogen metabolism is controlled by σ^{N}, but other functions of σ^{N}-dependent genes can be found in several organisms. Virulence genes are regulated by σ^{N} in multiple bacterial species. For example, in H. pylori, C. jejuni, and V. cholerae, class II flagellar genes (fliA and structural) are regulated by σ^{N} in a flagella virulence mechanism. In P. syringae and E. carotovora, hrpL gene is regulated by σ^{N} in a Type III secretion virulence mechanism. In P. aeruginosa, class II flagellar genes (regulatory and structural) are regulated by σ^{N} in a flagella virulence mechanism; algD and algC are regulated by σ^{N} in an alginate virulence mechanism; and pilA gene is regulated by σ^{N} in a pili virulence mechanism. Kazmierczak et al., Microbiology and Molecular Biology Reviews 69: 527-543, 2005.

“Protective immunity” or “protective immune response,” are intended to mean that the host mammal or host bird mounts an active immune response to an antigenic component of bacterial flagella, such that upon subsequent exposure to the Gram-negative bacteria or bacterial challenge, the mammal or bird is able to combat the infection. Thus, a protective immune response will decrease the incidence of morbidity and mortality from subsequent exposure to the Gram-negative bacteria among host mammals or host birds.

A protective immune response will also decrease colonization by the Gram-negative bacteria in the host birds. In this manner, transmission of infectious Gram-negative bacteria from avian species to mammalian species will be decreased and controlled. Those skilled in the art will understand that in a commercial poultry setting, the production of a protective immune response can be assessed by evaluating the effects of vaccination on the flock as a whole, e.g., there can still be morbidity and mortality in individual vaccinated birds.

“Active immune response” refers to an immunogenic response of the subject to an antigen. In particular, this term is intended to mean any level of protection from subsequent exposure to Gram-negative bacteria or bacterial antigens which is of some benefit in a population of subjects, whether in the form of decreased mortality, decreased lesions, improved feed conversion ratios, or the reduction of any other detrimental effect of the disease, and the like, regardless of whether the protection is partial or complete. An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development cell-mediated reactivity, or both.” Herbert B. Herscowitz, “Immunophysiology: Cell Function and Cellular Interactions in Antibody Formation,” in Immunology: Basic Processes 117 (Joseph A. Bellanti ed., 1985). Alternatively stated, an active immune response is mounted by the host after exposure to immunogens by infection, or as in the present case, by vaccination. Active immunity can be contrasted with passive immunity, which is acquired through the “transfer or preformed substances (antibody, transfer factor, thymic graft, interleukin-2) from an actively immunized host to a non-immune host.” Id.

“Inhibitors,” “activators,” and “modulators” of bacterial flagellar protein biosynthesis in cells are used to refer to inhibitory, activating, or modulating molecules, respectively, identified using in vitro and in vivo assays for bacterial flagellar protein biosynthesis, e.g., ligands, agonists, antagonists, and their homologs and mimetics.

“Modulator” includes inhibitors and activators. Inhibitors are agents that, e.g., bind to, partially or totally block stimulation, decrease, prevent, delay activation, inactivate, desensitize, or down regulate bacterial flagellar proteins or proteins involved in bacterial flagellar protein biosynthesis, e.g., antagonists. Activators are agents that, e.g., bind to, stimulate, increase, open, activate, facilitate, enhance activation, sensitize or up regulate the activity of proteins involved in bacterial flagellar protein biosynthesis, e.g., agonists. The proteins involved in bacterial flagellar protein biosynthesis include, but are not limited to, RpoN protein or FliA protein or structural protein FlgK. Modulators include agents that, e.g., alter the interaction of proteins involved in bacterial flagellar protein biosynthesis: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring proteins involved in bacterial flagellar protein biosynthesis, e.g., with altered activity, as well as naturally occurring and synthetic ligands, antagonists, agonists, small chemical molecules and the like. “Cell-based assays” for inhibitors and activators include, e.g., applying putative modulator compounds to a cell expressing a bacterial flagellar protein biosynthetic activity and then determining the functional effects on bacterial flagellar protein biosynthesis, as described herein. “Cell based assays” include, but are not limited to, in vivo tissue or cell samples from a mammalian subject or in vitro cell-based assays comprising proteins involved in bacterial flagellar protein biosynthesis that are treated with a potential activator, inhibitor, or modulator are compared to control samples without the inhibitor, activator, or modulator to examine the extent of inhibition. Control samples (untreated with inhibitors) can be assigned a relative bacterial flagellar protein biosynthetic activity value of 100%. Inhibition of bacterial flagellar protein biosynthesis is achieved when the bacterial flagellar protein biosynthetic activity value relative to the control is about 80%, optionally 50% or 25-0%. Activation of bacterial flagellar protein biosynthesis is achieved when the bacterial flagellar protein biosynthetic activity value relative to the control is 110%, optionally 150%, optionally 200-500%, or 1000-3000% higher.

The ability of a molecule to bind to proteins involved in bacterial flagellar protein biosynthesis can be determined, for example, by the ability of the putative ligand to bind to immunoadhesin (antibody to proteins involved in bacterial flagellar protein biosynthesis) coated on an assay plate. Specificity of binding can be determined by comparing binding to proteins not involved in bacterial flagellar protein biosynthesis.

“Test compound” refers to any compound tested as a modulator of proteins involved in bacterial flagellar protein biosynthesis. The test compound can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, test compound can be modulators that are genetically altered versions of proteins involved in bacterial flagellar protein biosynthesis. Typically, test compounds will be small organic molecules, peptides, lipids, or lipid analogs.

In one aspect, antibody binding to proteins, e.g., flgK protein, rpoN protein, or fliA protein, involved in bacterial flagellar protein biosynthesis can be assayed by either immobilizing the ligand or the receptor. For example, the assay can include immobilizing flgK protein, rpoN protein, or fliA protein fused to a His tag onto Ni-activated NTA resin beads. Antibody can be added in an appropriate buffer and the beads incubated for a period of time at a given temperature. After washes to remove unbound material, the bound protein can be released with, for example, SDS, buffers with a high pH, and the like and analyzed.

### Recombinant Nucleic Acid Techniques

The nucleic acids used to practice this invention, whether RNA, siRNA, antisense nucleic acid, cDNA, genomic DNA, vectors, viruses or hybrids thereof, can be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams, J. Am. Chem. Soc. 105: 661, 1983; Belousov, Nucleic Acids Res. 25: 3440-3444, 1997; Frenkel, Free Radic. Biol. Med. 19: 373-380, 1995; Blommers, Biochemistry 33: 7886-7896, 1994; Narang, Meth. Enzymol. 68: 90, 1979; Brown Meth. Enzymol. 68: 109, 1979; Beaucage, Tetra. Lett. 22: 1859, 1981; U.S. Pat. No. 4,458,066.

The invention provides oligonucleotides comprising sequences of the invention, e.g., subsequences of the exemplary sequences of the invention. Oligonucleotides can include, e.g., single stranded poly-deoxynucleotides or two complementary polydeoxynucleotide strands which can be chemically synthesized.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook and Russell, ed., MOLECULAR CLONING: A LABORATORY MANUAL (3rd ED.), Vols. 1-3, Cold Spring Harbor Laboratory, 2001; CURRENT PROTOCOLS 1N MOLECULAR BIOLOGY; Ausubel, ed. John Wiley & Sons, Inc., New York, 1997; LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y., 1993.

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, adioimmunoassay (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Obtaining and manipulating nucleic acids used to practice the methods of the invention can be done by cloning from genomic samples, and, if desired, screening and re-cloning inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld, Nat. Genet. 15: 333-335, 1997; yeast artificial chromosomes (YAC); bacterial artificial chromosomes (BAC); P1 artificial chromosomes, see, e.g., Woon, Genomics 50: 306-316, 1998; P1-derived vectors (PACs), see, e.g., Kern, Biotechniques 23:120-124, 1997; cosmids, recombinant viruses, phages or plasmids.

The invention provides fusion proteins and nucleic acids encoding them. A flgK protein, rpoN protein, or fliA protein can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams, Biochemistry 34: 1787-1797, 1995; Dobeli, Protein Expr. Purif 12: 404-414, 1998). The histidine residues facilitate detection and purification while the enterokinase cleavage site provides a means for purifying the epitope from the remainder of the fusion protein. In one aspect, a nucleic acid encoding a polypeptide is assembled in appropriate phase with a leader sequence capable of directing secretion of the translated polypeptide or fragment thereof. Technology pertaining to vectors encoding fusion proteins and application of fusion proteins are well described in the scientific and patent literature, see e.g., Kroll, DNA Cell. Biol. 12: 441-53, 1993.

A. Transcriptional Control Elements

The nucleic acids, as aspects of the invention, can be operatively linked to a promoter. A promoter can be one motif or an array of nucleic acid control sequences which direct transcription of a nucleic acid. A promoter can include necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. A “constitutive” promoter is a promoter which is active under most environmental and developmental conditions. An “inducible” promoter is a promoter which is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

B. Expression Vectors And Cloning Vehicles

Aspects of the invention provide expression vectors and cloning vehicles comprising nucleic acids of the invention, e.g., sequences encoding the proteins of the invention. Expression vectors and cloning vehicles can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors can include chromosomal, non-chromosomal and synthetic DNA sequences. Large numbers of suitable vectors are known to those of skill in the art, and are commercially available.

The nucleic acids of the invention can be cloned, if desired, into any of a variety of vectors using routine molecular biological methods; methods for cloning in vitro amplified nucleic acids are described, e.g., U.S. Pat. No. 5,426,039. To facilitate cloning of amplified sequences, restriction enzyme sites can be “built into” a PCR primer pair.

The invention provides libraries of expression vectors encoding polypeptides and peptides of the invention. These nucleic acids can be introduced into a genome or into the cytoplasm or a nucleus of a cell and expressed by a variety of conventional techniques, well described in the scientific and patent literature. See, e.g., Roberts, Nature 328: 731, 1987; Schneider, Protein Expr. Purif. 6435: 10, 1995; Sambrook, Tijssen or Ausubel. The vectors can be isolated from natural sources, obtained from such sources as ATCC or GenBank libraries, or prepared by synthetic or recombinant methods. For example, the nucleic acids of the invention can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required.

In one aspect, the nucleic acids of the invention are administered in vivo for in situ expression of the peptides or polypeptides of the invention. The nucleic acids can be administered as “naked DNA” (see, e.g., U.S. Pat. No. 5,580,859) or in the form of an expression vector, e.g., a recombinant virus. The nucleic acids can be administered by any route, including peri- or intra-tumorally, as described below. Vectors administered in vivo can be derived from viral genomes, including recombinantly modified enveloped or non-enveloped DNA and RNA viruses, preferably selected from baculoviridiae, parvoviridiae, picornoviridiae, herpesveridiae, poxyiridae, adenoviridiae, or picornnaviridiae. Chimeric vectors can also be employed which exploit advantageous merits of each of the parent vector properties (See e.g., Feng, Nature Biotechnology 15: 866-870, 1997). Such viral genomes can be modified by recombinant DNA techniques to include the nucleic acids of the invention; and can be further engineered to be replication deficient, conditionally replicating or replication competent. In alternative aspects, vectors are derived from the adenoviral (e.g., replication incompetent vectors derived from the human adenovirus genome, see, e.g., U.S. Pat. Nos. 6,096,718; 6,110,458; 6,113,913; 5,631,236); adeno-associated viral and retroviral genomes. Retroviral vectors can include those based upon murine leukemia virus (MuLV), gibbon ape leukemia virus (GaLV), Simian Immuno deficiency virus (SIV), human immuno deficiency virus (HIV), and combinations thereof, see, e.g., U.S. Pat. Nos. 6,117,681; 6,107,478; 5,658,775; 5,449,614; Buchscher, J. Virol. 66: 2731-2739, 1992; Johann, J. Virol. 66: 1635-1640, 1992). Adeno-associated virus (AAV)-based vectors can be used to adioimmun cells with target nucleic acids, e.g., in the in vitro production of nucleic acids and peptides, and in in vivo and ex vivo gene therapy procedures; see, e.g., U.S. Pat. Nos. 6,110,456; 5,474,935; Okada, Gene Ther. 3: 957-964, 1996.

“Expression cassette” as used herein refers to a nucleotide sequence which is capable of affecting expression of a structural gene (i.e., a protein coding sequence, such as a polypeptide of the invention) in a host compatible with such sequences. Expression cassettes include at least a promoter operably linked with the polypeptide coding sequence; and, optionally, with other sequences, e.g., transcription termination signals. Additional factors necessary or helpful in effecting expression can also be used, e.g., enhancers.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination. Thus, expression cassettes also include plasmids, expression vectors, recombinant viruses, any form of recombinant “naked DNA” vector, and the like.

“Vector” is intended to refer to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

C. Host Cells and Transformed Cells

The invention also provides a transformed cell comprising a nucleic acid sequence of the invention, e.g., a sequence encoding a polypeptide of the invention, or a vector of the invention. The host cell can be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter can be induced by appropriate means (e.g., temperature shift or chemical induction) and the cells can be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector can be glycosylated or can be non-glycosylated. Polypeptides of the invention can or can not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct can be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

D. Amplification of Nucleic Acids

In practicing the invention, nucleic acids encoding the polypeptides of the invention, or modified nucleic acids, can be reproduced by, e.g., amplification. The invention provides amplification primer sequence pairs for amplifying nucleic acids encoding polypeptides of the invention, e.g., primer pairs capable of amplifying nucleic acid sequences comprising the flgK protein, rpoN protein, or fliA protein, or subsequences thereof.

Amplification methods include, e.g., polymerase chain reaction, PCR (PCR PROTOCOLS, A GUIDE TO METHODS AND APPLICATIONS, ed. Innis, Academic Press, N.Y., 1990 and PCR STRATEGIES, 1995, ed. Innis, Academic Press, Inc., N.Y., ligase chain reaction (LCR) (see, e.g., Wu, Genomics 4: 560, 1989; Landegren, Science 241: 1077, 1988; Barringer, Gene 89: 117, 1990); transcription amplification (see, e.g., Kwoh, Proc. Natl. Acad. Sci. USA 86: 1173, 1989); and, self-sustained sequence replication (see, e.g., Guatelli, Proc. Natl. Acad. Sci. USA 87: 1874, 1990); Q Beta replicase amplification (see, e.g., Smith, J. Clin. Microbiol. 35: 1477-1491, 1997), automated Q-beta replicase amplification assay (see, e.g., Burg, Mol. Cell. Probes 10: 257-271, 1996) and other RNA polymerase mediated techniques (e.g., NASBA, Cangene, Mississauga, Ontario); see also Berger, Methods Enzymol. 152: 307-316, 1987; Sambrook; Ausubel; U.S. Pat. Nos. 4,683,195 and 4,683,202; Sooknanan, Biotechnology 13: 563-564, 1995.

E. Hybridization of Nucleic Acids

The invention provides isolated or recombinant nucleic acids that hybridize under stringent conditions to an exemplary sequence of the invention, e.g., a FlgK, RpoN, or FliA sequence, or the complement of any thereof, or a nucleic acid that encodes a polypeptide of the invention. In alternative aspects, the stringent conditions are highly stringent conditions, medium stringent conditions or low stringent conditions, as known in the art and as described herein. These methods can be used to isolate nucleic acids of the invention.

In alternative aspects, nucleic acids of the invention as defined by their ability to hybridize under stringent conditions can be between about five residues and the full length of nucleic acid of the invention; e.g., they can be at least 5, 10, 15, 20, 25, 30, 35, 40, 50, 55, 60, 65, 70, 75, 80, 90, 100, 150, 200, 250, 300, 350, 400, 450, 500, 550, 600, 650, 700, 750, 800 or more residues in length, or, the full length of a gene or coding sequence, e.g., cDNA. Nucleic acids shorter than full length are also included. These nucleic acids can be useful as, e.g., hybridization probes, labeling probes, PCR oligonucleotide probes, iRNA, antisense or sequences encoding antibody binding peptides (epitopes), motifs, active sites and the like.

“Selectively (or specifically) hybridizes to” refers to the binding, duplexing, or hybridizing of a molecule to a particular nucleotide sequence under stringent hybridization conditions when that sequence is present in a complex mixture (e.g., total cellular or library DNA or RNA), wherein the particular nucleotide sequence is detected at least at about 10 times background. In one aspect, a nucleic acid can be determined to be within the scope of the invention by its ability to hybridize under stringent conditions to a nucleic acid otherwise determined to be within the scope of the invention (such as the exemplary sequences described herein).

“Stringent hybridization conditions” refers to conditions under which a probe will hybridize to its target subsequence, typically in a complex mixture of nucleic acid, but not to other sequences in significant amounts (a positive signal (e.g., identification of a nucleic acid of the invention) is about 10 times background hybridization). Stringent conditions are sequence-dependent and will be different in different circumstances. Longer sequences hybridize specifically at higher temperatures. An extensive guide to the hybridization of nucleic acids is found in e.g., Sambrook and Russell, ed., MOLECULAR CLONING: A LABORATORY MANUAL (3rd ED.), Vols. 1-3, Cold Spring Harbor Laboratory, 2001; CURRENT PROTOCOLS 1N MOLECULAR BIOLOGY; Ausubel, ed. John Wiley & Sons, Inc., New York, 1997; LABORATORY TECHNIQUES IN BIOCHEMISTRY AND MOLECULAR BIOLOGY: HYBRIDIZATION WITH NUCLEIC ACID PROBES, PART I. Theory and Nucleic Acid Preparation, Tijssen, ed. Elsevier, N.Y., 1993.

Generally, stringent conditions are selected to be about 5-10° C. lower than the thermal melting point I for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30oC for short probes (e.g., 10 to 50 nucleotides) and at least about 60oC for long probes (e.g., greater than 50 nucleotides). Stringent conditions can also be achieved with the addition of destabilizing agents such as formamide as described in Sambrook (cited above). For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. For selective or specific hybridization, a positive signal (e.g., identification of a nucleic acid of the invention) is about 10 times background hybridization. Stringent hybridization conditions that are used to identify nucleic acids within the scope of the invention include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. In the present invention, genomic DNA or cDNA comprising nucleic acids of the invention can be identified in standard Southern blots under stringent conditions using the nucleic acid sequences disclosed here. Additional stringent conditions for such hybridizations (to identify nucleic acids within the scope of the invention) are those which include a hybridization in a buffer of 40% formamide, 1 M NaCl, 1% SDS at 37° C.

However, the selection of a hybridization format is not critical—it is the stringency of the wash conditions that set forth the conditions which determine whether a nucleic acid is within the scope of the invention. Wash conditions used to identify nucleic acids within the scope of the invention include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68oC for 15 minutes; or, equivalent conditions. See Sambrook, Tijssen and Ausubel for a description of SSC buffer and equivalent conditions.

F. Oligonucleotides Probes and Methods for Using Them

The invention also provides nucleic acid probes for identifying nucleic acids encoding a polypeptide which is a modulator or inhibitor of bacterial flagellar protein biosynthesis or proteins, e.g., flgK protein, rpoN protein, or fliA protein, involved in bacterial flagellar protein biosynthesis. In one aspect, the probe comprises at least 10 consecutive bases of a nucleic acid of the invention. Alternatively, a probe of the invention can be at least about 5, 6, 7, 8, 9, 10, 15, 20, 25, 30, 35, 40, 45, 50, 60, 70, 80, 90, 100, 110, 120, 130, 150 or about 10 to 50, about 20 to 60 about 30 to 70, consecutive bases of a sequence as set forth in a nucleic acid of the invention. The probes identify a nucleic acid by binding and/or hybridization. The probes can be used in arrays of the invention, see discussion below. The probes of the invention can also be used to isolate other nucleic acids or polypeptides.

G. Determining the Degree of Sequence Identity

The invention provides nucleic acids having at least 90%, 91%, 92%, 93%, 94%, 95%, 96%, 97%, 98%, 99% or more sequence identity to FlgK, RpoN, or FliA polynucleotide. The invention provides polypeptides having at least 90%, 91%, 92%, 93%, 94%, 95%, 96%, 97%, 98%, 99% or more sequence identity to flgK protein, rpoN protein, or fliA protein. The sequence identities can be determined by analysis with a sequence comparison algorithm or by a visual inspection. Protein and/or nucleic acid sequence identities (or homologies) can be evaluated using any of the variety of sequence comparison algorithms and programs known in the art.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.2.2. or FASTA version 3.0t78 algorithms and the default parameters discussed below can be used.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2: 482, 1981, by the homology alignment algorithm of Needleman & Wunsch, J. Mol. Biol. 48: 443, 1970, by the search for similarity method of Pearson & Lipman, Proc. Natl. Acad. Sci. U.S.A. 85: 2444, 1988, by computerized implementations of these algorithms (FASTDB (Intelligenetics), BLAST (National Center for Biomedical Information), GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., (1999 Suppl.), Current Protocols in Molecular Biology, Greene Publishing Associates and Wiley Interscience, N.Y., 1987)

A preferred example of an algorithm that is suitable for determining percent sequence identity and sequence similarity is the FASTA algorithm, which is described in Pearson & Lipman, Proc. Natl. Acad. Sci. U.S.A. 85: 2444, 1988. See also Pearson, Methods Enzymol. 266: 227-258, 1996. Preferred parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

Another preferred example of algorithm that is suitable for determining percent sequence identity and sequence similarity are the BLAST and BLAST 2.0 algorithms, which are described in Altschul et al., Nuc. Acids Res. 25: 3389-3402, 1977; and Altschul et al., J. Mol. Biol. 215: 403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. U.S.A. 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Natl. Acad. Sci. U.S.A. 90: 5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35: 351-360, 1987. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12: 387-395, 1984.

Another preferred example of an algorithm that is suitable for multiple DNA and amino acid sequence alignments is the CLUSTALW program (Thompson et al., Nucl. Acids. Res. 22: 4673-4680, 1994). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff, Proc. Natl. Acad. Sci. U.S.A. 89: 10915-10919, 1992).

“Sequence identity” refers to a measure of similarity between amino acid or nucleotide sequences, and can be measured using methods known in the art, such as those described below:

“Identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, 91%, 92%, 93%, 94%, 95%, 96%, 97%, 98%, or 99% or more identity over a specified region, when compared and aligned for maximum correspondence over a comparison window, or designated region as measured using one of the following sequence comparison algorithms or by manual alignment and visual inspection.

“Substantially identical,” in the context of two nucleic acids or polypeptides, refers to two or more sequences or subsequences that have at least of at least 60%, often at least 70%, preferably at least 80%, most preferably at least 90% or at least 95% nucleotide or amino acid residue identity, when compared and aligned for maximum correspondence, as measured using one of the following sequence comparison algorithms or by visual inspection. Preferably, the substantial identity exists over a region of the sequences that is at least about 50 bases or residues in length, more preferably over a region of at least about 100 bases or residues, and most preferably the sequences are substantially identical over at least about 150 bases or residues. In a most preferred aspect, the sequences are substantially identical over the entire length of the coding regions.

“Homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence (an exemplary sequence of FlgK, RpoN, or FliA gene product or flgK polypeptide, rpoN polypeptide, or fliA polypeptide) to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the numbers of contiguous residues. For example, in alternative aspects of the invention, continugous residues ranging anywhere from 20 to the full length of an exemplary polypeptide or nucleic acid sequence of the invention, e.g., FlgK, RpoN, or FliA polynucleotide or flgK polypeptide, rpoN polypeptide, or fliA polypeptide, are compared to a reference sequence of the same number of contiguous positions after the two sequences are optimally aligned. If the reference sequence has the requisite sequence identity to an exemplary polypeptide or nucleic acid sequence of the invention, e.g., at least 90%, 91%, 92%, 93%, 94%, 95%, 96%, 97%, 98%, 99% or more sequence identity to FlgK, RpoN, or FliA polynucleotide or flgK polypeptide, rpoN polypeptide, or fliA polypeptide, that sequence is within the scope of the invention.

Motifs which can be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

H. Computer Systems and Computer Program Products

To determine and identify sequence identities, structural homologies, motifs and the like in silico, the sequence of the invention can be stored, recorded, and manipulated on any medium which can be read and accessed by a computer. Accordingly, the invention provides computers, computer systems, computer readable mediums, computer programs products and the like recorded or stored thereon the nucleic acid and polypeptide sequences of the invention. As used herein, the words “recorded” and “stored” refer to a process for storing information on a computer medium. A skilled artisan can readily adopt any known methods for recording information on a computer readable medium to generate manufactures comprising one or more of the nucleic acid and/or polypeptide sequences of the invention.

Another aspect of the invention is a computer readable medium having recorded thereon at least one nucleic acid and/or polypeptide sequence of the invention. Computer readable media include magnetically readable media, optically readable media, electronically readable media and magnetic/optical media. For example, the computer readable media can be a hard disk, a floppy disk, a magnetic tape, CD-ROM, Digital Versatile Disk (DVD), Random Access Memory (RAM), or Read Only Memory (ROM) as well as other types of other media known to those skilled in the art.

As used herein, the terms “computer,” “computer program” and “processor” are used in their broadest general contexts and incorporate all such devices.

Vaccine Compositions of flgK Protein, rpoN Protein, or fliA Protein Antigens

FlgK protein, RpoN protein, or FliA protein sequences can be used to design oligonucleotide probes and used to screen genomic or cDNA libraries for genes from other Campylobacter jejuni serotypes. The basic strategies for preparing oligonucleotide probes and DNA libraries, as well as their screening by nucleic acid hybridization, are well known to those of ordinary skill in the art. See, e.g., DNA Cloning: Vol. I, supra; Nucleic Acid Hybridization, supra; Oligonucleotide Synthesis, supra; Sambrook et al., supra. Once a clone from the screened library has been identified by positive hybridization, it can be confirmed by restriction enzyme analysis and DNA sequencing that the particular library insert contains an FlgK gene, RpoN gene, or FliA gene, or a homolog thereof. The genes can then be further isolated using standard techniques and, if desired, PCR approaches or restriction enzymes employed to delete portions of the full-length sequence.

Similarly, genes can be isolated directly from bacteria using known techniques, such as phenol extraction and the sequence further manipulated to produce any desired alterations. See, e.g., Sambrook et al., supra, for a description of techniques used to obtain and isolate DNA. Alternatively, DNA sequences encoding the proteins of interest can be prepared synthetically rather than cloned. The DNA sequences can be designed with the appropriate codons for the particular amino acid sequence. In general, one will select preferred codons for the intended host if the sequence will be used for expression. The complete sequence is assembled from overlapping oligonucleotides prepared by standard methods and assembled into a complete coding sequence. See, e.g., Edge (1981) Nature 292: 756; Nambair et al. (1984) Science 223: 1299; Jay et al. (1984) J. Biol. Chem. 259: 6311.

Once coding sequences for the desired proteins have been prepared or isolated, they can be cloned into any suitable vector or replicon. Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGV 1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), Ylp5 (Saccharomyces), YCpI9 (Saccharomyces) and bovine papilloma virus (mammalian cells). See, Sambrook et al., supra; DNA Cloning, supra; B. Perbal, supra. The gene can be placed under the control of a promoter, ribosome binding site (for bacterial expression) and, optionally, an operator (collectively referred to herein as “control” elements), so that the DNA sequence encoding the desired protein is transcribed into RNA in the host cell transformed by a vector containing this expression construction. The coding sequence can or can not contain a signal peptide or leader sequence. Leader sequences can be removed by the host in post-translational processing. See, e.g., U.S. Pat. Nos. 4,431,739; 4,425,437; 4,338,397.

Other regulatory sequences can also be desirable which allow for regulation of expression of the protein sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements can also be present in the vector, for example, enhancer sequences.

The control sequences and other regulatory sequences can be ligated to the coding sequence prior to insertion into a vector, such as the cloning vectors described above. Alternatively, the coding sequence can be cloned directly into an expression vector which already contains the control sequences and an appropriate restriction site.

In some cases it can be necessary to modify the coding sequence so that it can be attached to the control sequences with the appropriate orientation; i.e., to maintain the proper reading frame. It can also be desirable to produce mutants or analogs of the protein. Mutants or analogs can be prepared by the deletion of a portion of the sequence encoding the protein, by insertion of a sequence, and/or by substitution of one or more nucleotides within the sequence. Techniques for modifying nucleotide sequences, such as site-directed mutagenesis, are described in, e.g., Sambrook et al., supra; DNA Cloning, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Madin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include, but are not limited to, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, but are not limited to, Aedes aegypti, Autographa californica, Bombyx mori, Drosophila melanogaster, Spodoptera fmgiperda, and Trichoplusia ni.

Depending on the expression system and host selected, the proteins of the present invention are produced by culturing host cells transformed by an expression vector described above under conditions whereby the protein of interest is expressed. The protein is then isolated from the host cells and purified. The selection of the appropriate growth conditions and recovery methods are within the skill of the art.

FlgK protein, RpoN protein, or FliA protein can also be produced by chemical synthesis such as solid phase peptide synthesis, using known amino acid sequences or amino acid sequences derived from the DNA sequence of the genes of interest. Such methods are known to those skilled in the art. See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides: Analysis, Synthesis, Biology, supra, Vol. 1, for classical solution synthesis. Chemical synthesis of peptides can be preferable if a small fragment of the antigen in question is capable of raising an immunological response in the subject of interest.

Once the above cell culture supernatants and, if desired, additional recombinant and/or purified proteins are produced, they are formulated into compositions for delivery to a mammalian subject. The vaccine composition is administered alone, or mixed with a pharmaceutically acceptable vehicle or excipient. Suitable vehicles are, for example, water, saline, dextrose, glycerol, ethanol, or the like, and combinations thereof. In addition, the vehicle can contain minor amounts of auxiliary substances such as wetting or emulsifying agents, pH buffering agents, or adjuvants in the case of vaccine compositions, which enhance the effectiveness of the vaccine. Suitable adjuvants are described further below. The compositions of the present invention can also include ancillary substances, such as pharmacological agents, cytokines, or other biological response modifiers.

Vaccine compositions in aspects of the present invention can include adjuvants to further increase the immunogenicity of one or more of the FlgK protein, RpoN protein, or FliA protein antigens. Such adjuvants include any compound or compounds that act to increase an immune response to an FlgK protein, RpoN protein, or FliA protein antigen or combination of antigens, thus reducing the quantity of antigen necessary in the vaccine, and/or the frequency of injection necessary in order to generate an adequate immune response. Adjuvants can include for example, emulsifiers, muramyl dipeptides, pyridine, aqueous adjuvants such as aluminum hydroxide, chitosan-based adjuvants, and any of the various saponins, oils, and other substances known in the art, such as Amphigen, LPS, bacterial cell wall extracts, bacterial DNA, synthetic oligonucleotides and combinations thereof (Schijns et al., Curr. Opi. Immunol. (2000) 12: 456), Mycobacterialphlei (M. phlei) cell wall extract (MCWE) (U.S. Pat. No. 4,744,984), M. phlei DNA (M-DNA), M-DNA-M. phlei cell wall complex (MCC). For example, compounds which can serve as emulsifiers herein include natural and synthetic emulsifying agents, as well as anionic, cationic and nonionic compounds. Among the synthetic compounds, anionic emulsifying agents include, for example, the potassium, sodium and ammonium salts of lauric and oleic acid, the calcium, magnesium and aluminum salts of fatty acids (i.e., metallic soaps), and organic sulfonates such as sodium lauryl sulfate. Synthetic cationic agents include, for example, cetyltrhethylammonlum bromide, while synthetic nonionic agents are exemplified by glycerylesters (e.g., glyceryl monostearate), polyoxyethylene glycol esters and ethers, and the sorbitan fatty acid esters (e.g., sorbitan monopalmitate) and their polyoxyethylene derivatives (e.g., polyoxyethylene sorbitan monopalmitate). Natural emulsifying agents include acacia, gelatin, lecithin and cholesterol.

Other suitable adjuvants can be formed with an oil component, such as a single oil, a mixture of oils, a water-in-oil emulsion, or an oil-in-water emulsion. The oil can be a mineral oil, a vegetable oil, or an animal oil. Mineral oil, or oil-in-water emulsions in which the oil component is mineral oil are preferred. In this regard, a “mineral oil” is defined herein as a mixture of liquid hydrocarbons obtained from petrolatum via a distillation technique; the term is synonymous with “liquid paraffin,” “liquid petrolatum” and “white mineral oil.” The term is also intended to include “light mineral oil,” i.e., an oil which is similarly obtained by distillation of petrolatum, but which has a slightly lower specific gravity than white mineral oil. See, e.g., Remington's Pharmaceutical Sciences, supra. A particularly preferred oil component is the oil-in-water emulsion sold under the trade name of EMULSIGEN PLUS™ (comprising a light mineral oil as well as 0.05% formalin, and 30 mcg/mL gentamicin as preservatives), available from MVP Laboratories, Ralston, Nebr. Suitable animal oils include, for example, cod liver oil, halibut oil, menhaden oil, orange roughy oil and shark liver oil, all of which are available commercially. Suitable vegetable oils, include, without limitation, canola oil, almond oil, cottonseed oil, corn oil, olive oil, peanut oil, safflower oil, sesame oil, soybean oil, and the like.

Alternatively, a number of aliphatic nitrogenous bases can be used as adjuvants with the vaccine formulations. For example, known immunologic adjuvants include mines, quaternary ammonium compounds, guanidines, benzamidines and thiouroniums (Gall, D. (1966) Immunology 11: 369-386). Specific compounds include dimethyldioctadecylammoniumbromide (DDA) (available from Kodak) and N,N-dioctadecyl-N,N-bis(2-hydroxyethyl)propanediine (“pyridine”). The use of DDA as an immunologic adjuvant has been described; see, e.g., the Kodak Laboratory Chemicals Bulletin 56(1): 1-5 (1986); Adv. Drug Deliv. Rev. 5(3):163-187 (1990); J. Controlled Release 7: 123-132 (1988); Clin. Exp. Immunol. 78(2): 256-262 (1989); J. Immunol. Methods 97(2): 159-164 (1987); Immunology 58(2): 245-250 (1986); and Int. Arch. Allergy Appl. Immunol. 68(3): 201-208 (1982). Avridine is also a well-known adjuvant. See, e.g U.S. Pat. No. 4,310,550 to Wolff, III et al., which describes the use of N,N-higher alkyl-N′,N′-bis(2-hydroxyethyl)propane diamines in general, and pyridine in particular, as vaccine adjuvants. U.S. Pat. No. 5,151,267 to Babiuk, and Babiuk et al. (1986) Virology 159: 57-66, also relate to the use of pyridine as a vaccine adjuvant.

An adjuvant for use with the vaccine is “VSA3” which is a modified form of the EMULSIGEN PLUS™ adjuvant which includes DDA (see, U.S. Pat. No. 5,951,988, incorporated herein by reference in its entirety).

Vaccine compositions including one or more of the FlgK protein, RpoN protein, or FliA protein antigens in aspects of the present invention can be prepared by uniformly and intimately bringing into association the vaccine composition preparations and the adjuvant using techniques well known to those skilled in the art including, but not limited to, mixing, sonication and microfluidation. The adjuvant will preferably comprise about 10 to 50% (v/v) of the vaccine, more preferably about 20 to 40% (v/v) and most preferably about 20 to 30% or 35% (v/v), or any integer within these ranges.

The compositions of aspects of the present invention are normally prepared as injectables, either as liquid solutions or suspensions, or as solid forms which are suitable for solution or suspension in liquid vehicles prior to injection. The preparation can also be prepared in solid form, emulsified or the active ingredient encapsulated in liposome vehicles or other particulate carriers used for sustained delivery. For example, the vaccine can be in the form of an oil emulsion, water in oil emulsion, water-in-oil-in-water emulsion, site-specific emulsion, long-residence emulsion, stickyemulsion, microemulsion, nanoemulsion, liposome, microparticle, microsphere, nanosphere, nanoparticle and various natural or synthetic polymers, such as nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures, that allow for sustained release of the vaccine.

Furthermore, the vaccine compositions including, for example, one or more of the FlgK protein, RpoN protein, or FliA protein antigens can be formulated into compositions in either neutral or salt forms. Pharmaceutically acceptable salts include the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Actual methods of preparing such dosage forms are known, or will be apparent, to those skilled in the art. See, e.g., Remington's Pharmaceutical Sciences, Mack Publishing Company, Easton, Pa., 18th edition, 1990.

The composition is formulated to contain an effective amount of FlgK protein antigen, RpoN protein antigen, or FliA protein antigen, the exact amount being readily determined by one skilled in the art, wherein the amount depends on the animal to be treated and the capacity of the animal's immune system to synthesize antibodies. The composition or formulation to be administered will contain a quantity of one or more secreted FlgK protein, RpoN protein, or FliA protein antigens adequate to achieve the desired state in the subject being treated. For purposes of the present invention, a therapeutically effective amount of a vaccine comprising a cell extract of FlgK protein, RpoN protein, or FliA protein antigents with or without added recombinant and/or purified secreted FlgK protein, RpoN protein, or FliA protein antigens, contains about 0.05 to 1500 μg secreted protein antigen, preferably about 10 to 1000 μg secreted protein antigen, more preferably about 30 to 500 μg and most preferably about 40 to 300 pg, or any integer between these values. FlgK protein, RpoN protein, or FliA protein antigens, can comprise about 10% to 50% of total cell extract protein, such as about 15% to 40% and most preferably about 15% to 25%. If supplemented with recombinant FlgK protein, RpoN protein, or FliA protein, the vaccine can contain about 5 to 500 μg of protein, more preferably about 10 to 250 μg and most preferably about 20 to 125 μg.

Routes of administration include, but are not limited to, oral, topical, subcutaneous, intramuscular, intravenous, subcutaneous, intradermal, transdermal and subdermal. Depending on the route of administration, the volume per dose is preferably about 0.001 to 10 ml, more preferably about 0.01 to 5 ml, and most preferably about 0.1 to 3 ml. Vaccine can be administered in a single dose treatment or in multiple dose treatments (boosts) on a schedule and over a time period appropriate to the age, weight and condition of the subject, the particular vaccine formulation used, and the route of administration.

Any suitable pharmaceutical delivery means can be employed to deliver the compositions to the vertebrate subject, e.g., an avian subject or mammalian subject. For example, conventional needle syringes, spring or compressed gas (air) injectors (U.S. Pat. Nos. 1,605,763 to Smoot; 3,788,315 to Laurens; 3,853,125 to Clark et al; 4,596,556 to Morrow et al.; and 5,062,830 to Dunlap), liquid jet injectors (U.S. Pat. Nos. 2,754,818 to Scherer; 3,330,276 to Gordon; and 4,518,385 to Lindcaner et al.), and particle injectors (U.S. Pat. Nos. 5,149,655 to McCabe et al. and 5,204,253 to Sanford et al.) are all appropriate for delivery of the compositions.

If a jet injector is used, a single jet of the liquid vaccine composition is ejected under high pressure and velocity, e.g., 1200-1400 PSI, thereby creating an opening in the skin and penetrating to depths suitable for immunization.

Peptide Mimetics of flgK Protein, rpoN Protein, or fliA Protein

The antigenic bacterial flagellar protein or protein involved in bacterial flagellar protein biosynthesis administered as a vaccine composition of the present invention, or any portion thereof of the protein, can be modified to enhance their half life. Peptide analogs are commonly used in the pharmaceutical industry as non-peptide drugs with properties analogous to those of the template peptide. These types of non-peptide compounds are termed “peptide mimetics” or “peptidomimetics” (Fauchere, Adv. Drug Res. 15: 29, 1986; Veber et al., TINS p. 392, 1985; and Evans et al., J. Med. Chem. 30: 1229, 1987, which are incorporated herein by reference) and are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), such as an antigen polypeptide, but have one or more peptide linkages optionally replaced by a linkage selected from the group consisting of:—CH₂NH—, —CH₂S—, —CH₂—CH₂—, —CH═CH—(cis and trans), —COCH₂—, —CH(OH)CH₂—, and —CH₂SO—, by methods known in the art and further described in the following references: Spatola, A. F. in Chemistry and Biochemistry of Amino Acids, Peptides, and Proteins Weinstein, B., ed., Marcel Dekker, New York, p. 267, 1983; Spatola, A. F., Vega Data, Vol. 1, Issue 3, “Peptide Backbone Modifications,” 1983; Morley, Trends. Pharm. Sci. pp. 463-468, 1980; Hudson et al., Int. J. Pept. Prot. Res. 14: 177-185, 1979 (—CH₂NH—, CH₂CH₂—); Spatola et al., Life. Sci. 38: 1243-1249, 1986 (—CH₂—S); Hann, J. Chem. Soc. Perkin. Trans. 1: 307-314, 1982 (—CH—CH—, cis and trans); Almquist et al., J. Med. Chem. 23: 1392-1398, 1980 (—COCH₂—); Jennings-White et al., Tetrahedron Lett. 23: 2533, 1982 (—COCH₂—); Szelke et al., European Patent Application No. EP 45665 CA: 97: 39405, 1982 (—CH(OH)CH₂—); Holladay et al., Tetrahedron. Lett. 24: 4401-4404, 1983 (—C(OH)CH₂—); and Hruby, Life Sci. 31: 189-199, 1982 (—CH₂—S—); each of which is incorporated herein by reference. A particularly preferred non-peptide linkage is —CH₂NH—. Such peptide mimetics can have significant advantages over polypeptide aspects, including, for example: more economical production, greater chemical stability, enhanced pharmacological properties (half-life, absorption, potency, efficacy), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivatization (e.g., labeling) of peptidomimetics should not substantially interfere with the desired biological or pharmacological activity of the peptidomimetic.

Systematic substitution of one or more amino acids of an amino acid sequence with a D-amino acid of the same type (e.g., D-lysine in place of L-lysine) can be used to generate more stable peptides. In addition, constrained peptides can be generated by methods known in the art (Rizo et al., Annu. Rev. Biochem. 61: 387, 1992, incorporated herein by reference); for example, by adding internal cysteine residues capable of forming intramolecular disulfide bridges which cyclize the peptide.

Such modified polypeptides can be produced in prokaryotic or eukaryotic host cells. Alternatively, such peptides can be synthesized by chemical methods. Methods for expression of heterologous polypeptides in recombinant hosts, chemical synthesis of polypeptides, and in vitro translation are well known in the art and are described further in Maniatis et al., Molecular Cloning: A Laboratory Manual, 2nd Ed., Cold Spring Harbor, N.Y., 1989; Berger et al., Methods in Enzymology, Volume 152, Guide to Molecular Cloning Techniques, 1987, Academic Press, Inc., San Diego, Calif.; Merrifield, J. Am. Chem. Soc. 91: 501, 1969; Chaiken, CRC Crit. Rev. Biochem. 11: 255, 1981; Kaiser et al., Science 243: 187, 1989; Merrifield, Science 232: 342, 1986; Kent, Annu. Rev. Biochem. 57: 957, 1988; and Offord, Semisynthetic Proteins, Wiley Publishing, 1980, which are incorporated herein by reference).

Polypeptides can be produced, typically by direct chemical synthesis, and used as a binding moiety of a heteropolymer. Peptides can be produced as modified peptides, with nonpeptide moieties attached by covalent linkage to the N-terminus and/or C-terminus. In certain preferred aspects, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal modifications such as amidation, as well as other terminal modifications, including cyclization, can be incorporated into various aspects of the test compounds. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others.

Antibodies as Modulators of flgK Protein, rpoN Protein, or fliA Protein Activity or of Bacterial Flagellar Protein Biosynthetic Activity

The antibodies and antigen-binding fragments thereof described herein specifically bind to and/or inhibit bacterial flagellar protein biosynthetic activity or specifically bind to and/or inhibit flgK protein, rpoN protein, or fliA protein activity and can modulate or activate an immune response to Gram negative bacterial infection in a vertebrate, mammalian, or avian subject.

Antibodies that bind flgK protein, rpoN protein, or fliA protein are useful as compounds that modulate or inhibit signaling via a bacterial flagellar protein biosynthetic pathway in bacterial cells.

In some aspects, the antibody or antigen-binding fragment thereof or selectively binds (e.g., competitively binds, or binds to same epitope, e.g., a conformational or a linear epitope) to an antigen that is selectively bound by an antibody produced by a hybridoma cell line. Thus, the epitope can be in close proximity spatially or functionally-associated, e.g., an overlapping or adjacent epitope in linear sequence or conformational space, to a known epitope bound by an antibody. Potential epitopes can be identified computationally using a peptide threading program, and verified using methods known in the art, e.g., by assaying binding of the antibody to mutants or fragments of the FlgK, RpoN, or FliA gene product, e.g., mutants or fragments of a domain of flgK protein, rpoN protein, or fliA protein.

Methods of determining the sequence of an antibody described herein are known in the art; for example, the sequence of the antibody can be determined by using known techniques to isolate and identify a cDNA encoding the antibody from the hybridoma cell line. Methods for determining the sequence of a cDNA are known in the art.

The antibodies described herein typically have at least one or two heavy chain variable regions (V_{H}), and at least one or two light chain variable regions (V_{L}). The V_{H} and V_{L} regions can be further subdivided into regions of hypervariability, termed complementarity determining regions (CDR), which are interspersed with more highly conserved framework regions (FR). These regions have been precisely defined (see, Kabat et al., Sequences of Proteins of Immunological Interest, Fifth Edition, U.S. Department of Health and Human Services, NIH Publication No. 91-3242, 1991 and Chothia et al., J. Mol. Biol. 196: 901-917, 1987). Antibodies or antibody fragments containing one or more framework regions are also useful in the invention. Such fragments have the ability to specifically bind to a domain of flgK protein, rpoN protein, or fliA protein and to modulate or inhibit proteins involved in bacterial flagellar protein biosynthesis in a cell, or to modulate or inhibit an immune response to Gram negative bacteria.

An antibody as described herein can include a heavy and/or light chain constant region (constant regions typically mediate binding between the antibody and host tissues or factors, including effector cells of the immune system and the first component (C1q) of the classical complement system), and can therefore form heavy and light immunoglobulin chains, respectively. For example, the antibody can be a tetramer (two heavy and two light immunoglobulin chains, which can be connected by, for example, disulfide bonds). The antibody can contain only a portion of a heavy chain constant region (e.g., one of the three domains heavy chain domains termed C_{H}1, C_{H}2, and C_{H}3, or a portion of the light chain constant region (e.g., a portion of the region termed C_{L}).

Antigen-binding fragments are also included in the invention. Such fragments can be: (i) a F_{ab} fragment (i.e., a monovalent fragment consisting of the V_{L}, V_{H}, C_{L}, and C_{H}1 domains); (ii) a F(_{ab′)}₂ fragment (i.e., a bivalent fragment containing two F_{ab} fragments linked by a disulfide bond at the hinge region); (iii) a F_{d} fragment consisting of the V_{H} and C_{H}1 domains; (iv) a Fᵥ fragment consisting of the V_{L} and V_{H} domains of a single arm of an antibody, (v) a dAb fragment (Ward et al., Nature 341: 544-546, 1989), which consists of a V_{H} domain; and/or (vi) an isolated complementarity determining region (CDR).

Fragments of antibodies (including antigen-binding fragments as described above) can be synthesized using methods known in the art such as in an automated peptide synthesizer, or by expression of a full-length gene or of gene fragments in, for example, FlgK, RpoN, or FliA gene product F(_{ab}′)₂ fragments can be produced by pepsin digestion of an antibody molecule, and F_{ab} fragments can be generated by reducing the disulfide bridges of F(_{ab}′)₂ fragments. Alternatively, F_{ab} expression libraries can be constructed (Huse et al., Science 246: 1275-81, 1989) to allow relatively rapid identification of monoclonal F_{ab} fragments with the desired specificity.

Methods of making other antibodies and antibody fragments are known in the art. For example, although the two domains of the Fv fragment, V_{L} and V_{H}, are coded for by separate genes, they can be joined, using recombinant methods or a synthetic linker that enables them to be made as a single protein chain in which the V_{L} and V_{H} regions pair to form monovalent molecules (known as single chain Fv (scFv); see e.g., Bird et al., Science 242: 423-426, 1988; Huston et al., Proc. Natl. Acad. Sci. USA 85: 5879-5883, 1988; Colcher et al., Ann. NY Acad. Sci. 880: 263-80, 1999; and Reiter, Clin. Cancer Res. 2: 245-52, 1996).

Techniques for producing single chain antibodies are also described in U.S. Pat. Nos. 4,946,778 and 4,704,692. Such single chain antibodies are encompassed within the term “antigen-binding fragment” of an antibody. These antibody fragments are obtained using conventional techniques known to those of ordinary skill in the art, and the fragments are screened for utility in the same manner that intact antibodies are screened. Moreover, a single chain antibody can form complexes or multimers and, thereby, become a multivalent antibody having specificities for different epitopes of the same target protein.

Antibodies and portions thereof that are described herein can be monoclonal antibodies, generated from monoclonal antibodies, or can be produced by synthetic methods known in the art. Antibodies can be recombinantly produced (e.g., produced by phage display or by combinatorial methods, as described in, e.g., U.S. Pat. No. 5,223,409; WO 92/18619; WO 91/17271; WO 92/20791; WO 92/15679; WO 93/01288; WO 92/01047; WO 92/09690; WO 90/02809; Fuchs et al., Bio/Technology 9: 1370-1372, 1991; Hay et al., Human Antibody Hybridomas 3: 81-85, 1992; Huse et al., Science 246: 1275-1281, 1989; Griffiths et al., EMBO J. 12: 725-734, 1993; Hawkins et al., J. Mol. Biol. 226: 889-896, 1992; Clackson et al., Nature 352: 624-628, 1991; Gram et al., Proc. Natl. Acad. Sci. USA 89: 3576-3580, 1992; Garrad et al., Bio/Technology 9: 1373-1377, 1991; Hoogenboom et al., Nucl. Acids Res. 19: 4133-4137, 1991; and Barbas et al., Proc. Natl. Acad. Sci. USA 88: 7978-7982, 1991).

As one example, an antibody to proteins, e.g., flgK protein, rpoN protein, or fliA protein, involved in bacterial flagellar protein biosynthesis can be made by immunizing an animal with a flgK protein, rpoN protein, or fliA protein, or fragment e.g., an antigenic peptide fragment derived from or having the sequence of a portion of FlgK, RpoN, or FliA gene product thereof, or a cell expressing the flgK protein, rpoN protein, or fliA protein antigen or an antigenic fragment thereof. In some aspects, antibodies or antigen-binding fragments thereof described herein can bind to a purified FlgK, RpoN, or FliA gene product. In some aspects, the antibodies or antigen-binding fragments thereof can bind to a FlgK, RpoN, or FliA gene product in a tissue section, a whole cell (living, lysed, or fractionated), or a membrane fraction. Antibodies can be tested, e.g., in in vitro systems, such as measuring modulation or inhibition of FlgK, RpoN, or FliA gene expression or bacterial flagellar protein biosynthetic activity.

In the event an antigenic peptide derived from FlgK, RpoN, or FliA gene product is used, it will typically include at least eight (e.g., 10, 15, 20, 30, 50, 100 or more) consecutive amino acid residues of a domain of FlgK, RpoN, or FliA gene product. In some aspects, the antigenic peptide will comprise all of the domain of FlgK, RpoN, or FliA gene product. The antibodies generated can specifically bind to one of the proteins in their native form (thus, antibodies with linear or conformational epitopes are within the invention), in a denatured or otherwise non-native form, or both. Peptides likely to be antigenic can be identified by methods known in the art, e.g., by computer-based antigenicity-predicting algorithms. Conformational epitopes can sometimes be identified by identifying antibodies that bind to a protein in its native form, but not in a denatured form.

The host animal (e.g., a chicken or other avian species, a human, rabbit, mouse, guinea pig, or rat) can be immunized with the antigen, optionally linked to a carrier (i.e., a substance that stabilizes or otherwise improves the immunogenicity of an associated molecule), and optionally administered with an adjuvant (see, e.g., Ausubel et al., supra). An exemplary carrier is keyhole limpet hemocyanin (KLH) and exemplary adjuvants, which will typically be selected in view of the host animal's species, include Freund's adjuvant (complete or incomplete), adjuvant mineral gels (e.g., aluminum hydroxide), surface active substances such as lysolecithin, pluronic polyols, polyanions, peptides, oil emulsions, dinitrophenol, BCG (bacille Calmette-Guerin), and Corynebacterium parvum. KLH is also sometimes referred to as an adjuvant. The antibodies generated in the host can be purified by, for example, affinity chromatography methods in which the polypeptide antigen or a fragment thereof, is immobilized on a resin.

These sequences can be used to design oligonucleotide probes and used to screen genomic or cDNA libraries for genes from other C. jejuni serotypes. The basic strategies for preparing oligonucleotide probes and DNA libraries, as well as their screening by nucleic acid hybridization, are well known to those of ordinary skill in the art. See, e.g., DNA Cloning: Vol. I, supra; Nucleic Acid Hybridization, supra; Oligonucleotide Synthesis, supra; Sambrook et al., supra. Once a clone from the screened library has been identified by positive hybridization, it can be confirmed by restriction enzyme analysis and DNA sequencing that the particular library insert contains a bacterial flagellar protein, e.g., FlgK protein, or a protein involved in bacterial flagellar protein biosynthesis gene, e.g., RpoN protein or FliA protein, or a homolog thereof. The genes can then be further isolated using standard techniques and, if desired, PCR approaches or restriction enzymes employed to delete portions of the full-length sequence.

Epitopes encompassed by an antigenic peptide will typically be located on the surface of the protein (e.g., in hydrophilic regions), or in regions that are highly antigenic (such regions can be selected, initially, by virtue of containing many charged residues). An Emini surface probability analysis of human protein sequences can be used to indicate the regions that have a particularly high probability of being localized to the surface of the protein.

The antibody can be a fully human antibody (e.g., an antibody made in a mouse or other mammal that has been genetically engineered to produce an antibody from a human immunoglobulin sequence, such as that of a human immunoglobulin gene (the kappa, lambda, alpha (IgA₁ and IgA₂), gamma (IgG₁, IgG₂, IgG₃, IgG₄), delta, epsilon and mu constant region genes or the myriad immunoglobulin variable region genes). Alternatively, the antibody can be a non-human antibody (e.g., a rodent (e.g., a mouse or rat), goat, rabbit, or non-human primate (e.g., monkey) antibody).

Human monoclonal antibodies can be generated in transgenic mice carrying the human immunoglobulin genes rather than those of the mouse. Splenocytes obtained from these mice (after immunization with an antigen of interest) can be used to produce hybridomas that secrete human mAbs with specific affinities for epitopes from a human protein (see, e.g., WO 91/00906, WO 91/10741; WO 92/03918; WO 92/03917; Lonberg et al., Nature 368: 856-859, 1994; Green et al., Nature Genet. 7: 13-21, 1994; Morrison et al., Proc. Natl. Acad. Sci. USA 81: 6851-6855, 1994; Bruggeman et al, Immunol. 7: 33-40, 1993; Tuaillon et al., Proc. Natl. Acad. Sci. USA 90: 3720-3724, 1993; and Bruggeman et al, Eur. J. Immunol. 21: 1323-1326, 1991).

The anti-flgK protein, anti-rpoN protein, or anti-fliA protein antibody can also be one in which the variable region, or a portion thereof (e.g., a CDR), is generated in a non-human organism (e.g., a rat or mouse). Thus, the invention encompasses chimeric, CDR-grafted, and humanized antibodies and antibodies that are generated in a non-human organism and then modified (m, e.g., the variable framework or constant region) to decrease antigenicity in a human. Chimeric antibodies (i.e., antibodies in which different portions are derived from different animal species (e.g., the variable region of a murine m-Ab and the constant region of a human immunoglobulin) can be produced by recombinant techniques known in the art. For example, a gene encoding the F_{c} constant region of a murine (or other species) monoclonal antibody molecule can be digested with restriction enzymes to remove the region encoding the murine F_{c}, and the equivalent portion of a gene encoding a human F_{c} constant region can be substituted therefore (see, e.g., European Patent Application Nos. 125,023; 184,187; 171,496; and 173,494; see also WO 86/01533; U.S. Pat. No. 4,816,567; Better et al., Science 240: 1041-1043, 1988; Liu et al., Proc. Natl. Acad. Sci. USA 84: 3439-3443, 1987; Liu et al., J. Immunol. 139: 3521-3526, 1987; Sun et al., Proc. Natl. Acad. Sci. USA 84: 214-218, 1987; Nishimura et al., Cancer Res. 47: 999-1005, 1987; Wood et al, Nature 314: 446-449, 1985; Shaw et al., J. Natl. Cancer Inst. 80: 1553-1559, 1988; Morrison et al., Proc. Natl. Acad. Sci. USA 81: 6851, 1984; Neuberger et al., Nature 312: 604, 1984; and Takeda et al., Nature 314: 452, 1984).

In a humanized or CDR-grafted antibody, at least one or two, but generally all three of the recipient CDRs (of heavy and or light immunoglobulin chains) will be replaced with a donor CDR (see, e.g., U.S. Pat. No. 5,225,539; Jones et al., Nature 321: 552-525, 1986; Verhoeyan et al., Science 239: 1534, 1988; and Beidler et al., J. Immunol. 141: 4053-4060, 1988). One need replace only the number of CDRs required for binding of the humanized antibody to FlgK, RpoN, or FliA gene product. The donor can be a rodent antibody, and the recipient can be a human framework or a human consensus framework. Typically, the immunoglobulin providing the CDRs is called the “donor” (and is often that of a rodent) and the immunoglobulin providing the framework is called the “acceptor.” The acceptor framework can be a naturally occurring (e.g., a human) framework, a consensus framework or sequence, or a sequence that is at least 85% (e.g., 90%, 95%, 99%) identical thereto. A “consensus sequence” is one formed from the most frequently occurring amino acids (or nucleotides) in a family of related sequences (see, e.g., Winnaker, From Genes to Clones, Verlagsgesellschaft, Weinheim, Germany, 1987). Each position in the consensus sequence is occupied by the amino acid residue that occurs most frequently at that position in the family (where two occur equally frequently, either can be included). A “consensus framework” refers to the framework region in the consensus immunoglobulin sequence. Humanized antibodies to FlgK, RpoN, or FliA gene product can be made in which specific amino acid residues have been substituted, deleted or added (m, e.g., in the framework region to improve antigen binding). For example, a humanized antibody will have framework residues identical to those of the donor or to amino acid a receptor other than those of the recipient framework residue. To generate such antibodies, a selected, small number of acceptor framework residues of the humanized immunoglobulin chain are replaced by the corresponding donor amino acids. The substitutions can occur adjacent to the CDR or in regions that interact with a CDR (U.S. Pat. No. 5,585,089, see especially columns 12-16). Other techniques for humanizing antibodies are described in EP 519596 A1.

An antibody to FlgK, RpoN, or FliA gene product can be humanized as described above or using other methods known in the art. For example, humanized antibodies can be generated by replacing sequences of the Fv variable region that are not directly involved in antigen binding with equivalent sequences from human Fv variable regions. General methods for generating humanized antibodies are provided by Morrison, Science 229: 1202-1207, 1985; Oi et al., BioTechniques 4: 214, 1986, and Queen et al. (U.S. Pat. Nos. 5,585,089; 5,693,761, and 5,693,762). The nucleic acid sequences required by these methods can be obtained from a hybridoma producing an antibody against FlgK, RpoN, or FliA or fragments thereof having the desired properties such as the ability to measure modulation or inhibition of FlgK, RpoN, or FliA gene expression or bacterial flagellar protein biosynthesis resulting in inhibition of Gram negative bacterial infection. The recombinant DNA encoding the humanized antibody, or fragment thereof, can then be cloned into an appropriate expression vector.

In certain aspects, the antibody has an effector function and can fix complement, while in others it can neither recruit effector cells nor fix complement. The antibody can also have little or no ability to bind an Fc receptor. For example, it can be an isotype or subtype, or a fragment or other mutant that cannot bind to an Fc receptor (e.g., the antibody can have a mutant (e.g., a deleted) Fc receptor binding region). Antibodies lacking the Fc region typically cannot fix complement, and thus are less likely to cause the death of the cells they bind to.

In other aspects, the antibody can be coupled to a heterologous substance, such as a therapeutic agent (e.g., an antibiotic), or a detectable label. A detectable label can include an enzyme (e.g., horseradish peroxidase, alkaline phosphatase, .beta.-galactosidase, or acetylcholinesterase), a prosthetic group (e.g., streptavidin/biotin and avidin/biotin), or a fluorescent, luminescent, bioluminescent, or radioactive material (e.g., umbelliferone, fluorescein, fluorescein isothiocyanate, rhodamine, dichlorotriazinylamine fluorescein, dansyl chloride or phycoerythrin (which are fluorescent), luminol (which is luminescent), luciferase, luciferin, and aequorin (which are bioluminescent), and ⁹⁹ mTc, ¹⁸Re, ¹¹¹In, ¹²⁵I, ¹³¹I, ³⁵S or ³H (which are radioactive)).

The antibodies described herein (e.g., monoclonal antibodies) can also be used to isolate flgK protein, rpoN protein, or fliA protein or fragments thereof such as the fragment associated with modulation or inhibition of FlgK, RpoN, or FliA gene expression or bacterial flagellar protein biosynthesis resulting in inhibition of Gram negative bacterial infection (by, for example, affinity chromatography or immunoprecipitation) or to detect them in, for example, a cell lysate or supernatant (by Western blotting, enzyme-linked immunosorbant assays (ELISAs), radioimmune assays, and the like) or a histological section. These methods permit the determination of the abundance and pattern of expression of a particular protein. This information can be useful in making a diagnosis or in evaluating the efficacy of a clinical test or treatment.

The invention also includes the nucleic acids that encode the antibodies described above and vectors and cells (e.g., mammalian cells such as CHO cells or lymphatic cells) that contain them (e.g., cells transformed with a nucleic acid that encodes an antibody that specifically binds to flgK protein, rpoN protein, or fliA protein). Similarly, the invention includes cell lines (e.g., hybridomas) that make the antibodies of the invention and methods of making those cell lines.

High Throughput Assays for Modulators of flgK Protein, rpoN Protein, or fliA Protein

The compounds tested as modulators of flgK protein, rpoN protein, or fliA protein can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, modulators can be genetically altered versions of flgK protein, rpoN protein, or fliA protein. Typically, test compounds will be small organic molecules, peptides, lipids, and lipid analogs.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred aspect, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37: 487-493, 1991 and Houghton et al., Nature 354: 84-88, 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90: 6909-6913, 1993), vinylogous polypeptides (Hagihara et al., J. Amer. Chem. Soc. 114: 6568, 1992), nonpeptidal peptidomimetics with glucose scaffolding (Hirschmann et al., J. Amer. Chem. Soc. 114: 9217-9218, 1992), analogous organic syntheses of small compound libraries (Chen et al., J. Amer. Chem. Soc. 116: 2661, 1994), oligocarbamates (Cho et al., Science 261: 1303, 1993), and/or peptidyl phosphonates (Campbell et al., J. Org. Chem. 59: 658, 1994), nucleic acid libraries (see Ausubel, Berger and Sambrook, all supra), peptide nucleic acid libraries (see, e.g., U.S. Pat. No. 5,539,083), antibody libraries (see, e.g., Vaughn et al, Nature Biotechnology, 14: 309-314, 1996 and PCT/US96/10287), carbohydrate libraries (see, e.g., Liang et al., Science 274: 1520-1522, 1996 and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, Ru, 3D Pharmaceuticals, Exton, Pa., Martek Biosciences, Columbia, Md.).

Candidate compounds are useful as part of a strategy to identify drugs for preventing or treating Gram-negative bacterial infection in a vertebrate subject by administering an antagonist of bacterial flagellar protein biosynthesis to the vertebrate subject in an amount effective to reduce or eliminate the bacterial infection. A test compound that binds to flgK protein, rpoN protein, or fliA protein and modulates or inhibits bacterial flagellar protein biosynthesis is considered a candidate compound.

Screening assays for identifying candidate or test compounds that bind to flgK protein, rpoN protein, or fliA protein, or modulate or inhibit the activity of flgK protein, rpoN protein, or fliA proteins or polypeptides or biologically active portions thereof, to inhibit bacterial flagellar protein biosynthesis, are also included in the invention. The test compounds can be obtained using any of the numerous approaches in combinatorial library methods known in the art, including, but not limited to, biological libraries; spatially addressable parallel solid phase or solution phase libraries; synthetic library methods requiring deconvolution; the “one-bead one-compound” library method; and synthetic library methods using affinity chromatography selection. The biological library approach can be used for, e.g., peptide libraries, while the other four approaches are applicable to peptide, non-peptide oligomer or small chemical molecule libraries of compounds (Lam, Anticancer Drug Des. 12: 145, 1997). Examples of methods for the synthesis of molecular libraries can be found in the art, for example in: DeWitt et al., Proc. Natl. Acad. Sci. U.S.A. 90: 6909, 1993; Erb et al., Proc. Natl. Acad. Sci. USA 91: 11422, 1994; Zuckermann et al., J. Med. Chem. 37: 2678, 1994; Cho et al., Science 261: 1303, 1993; Carrell et al., Angew. Chem. Int. Ed. Engl. 33: 2059, 1994; Carell et al., Angew. Chem. Int. Ed. Engl. 33: 2061, 1994; and Gallop et al., J. Med. Chem. 37: 1233, 1994. In some aspects, the test compounds are inhibiting variants of flgK protein, rpoN protein, or fliA protein.

Libraries of compounds can be presented in solution (e.g., Houghten, Bio/Techniques 13: 412-421, 1992), or on beads (Lam, Nature 354: 82-84, 1991), chips (Fodor, Nature 364: 555-556, 1993), bacteria (U.S. Pat. No. 5,223,409), spores (U.S. Pat. Nos. 5,571,698, 5,403,484, and 5,223,409), plasmids (Cull et al., Proc. Natl. Acad. Sci. USA 89: 1865-1869, 1992) or on phage (Scott et al., Science 249: 386-390, 1990; Devlin, Science 249: 404-406, 1990; Cwirla et al., Proc. Natl. Acad. Sci. USA 87: 6378-6382, 1990; and Felici, J. Mol. Biol. 222: 301-310, 1991).

The ability of a test compound to modulate the activity of bacterial flagellar protein biosynthesis, e.g., of flgK protein, rpoN protein, or fliA protein or a biologically active portion thereof can be determined, e.g., by monitoring the ability to inhibit bacterial flagellar protein biosynthesis in the presence of the test compound. Modulating the activity of flgK protein, rpoN protein, or fliA protein or a biologically active portion thereof can be determined by measuring inhibition of bacterial flagellar protein biosynthesis. The ability of the test compound to modulate or inhibit the activity of flgK protein, rpoN protein, or fliA protein, or a biologically active portion thereof, can also be determined by monitoring the ability of proteins to inhibit bacterial flagellar protein biosynthesis. The binding assays can be cell-based or cell-free.

The ability of a test compound to prevent or treat Gram-negative bacterial infection in a vertebrate subject by administering an antagonist of bacterial flagellar protein biosynthesis to the vertebrate subject in an amount effective to reduce or eliminate the bacterial infection can be determined by one of the methods described herein or known in the art for determining direct binding of a test compound to flgK protein, rpoN protein, or fliA protein or other protein involved in bacterial flagellar protein biosynthesis. In one aspect, the ability of a test compound to inhibit bacterial flagellar protein biosynthesis by inhibiting flgK protein, rpoN protein, or fliA protein activity can be performed in an assay as described herein. Detection of inhibit bacterial flagellar protein biosynthesis can be developed by the expression of a recombinant flgK protein, rpoN protein, or fliA protein that also encodes a detectable marker such as a FLAG sequence or a luciferase. This assay can be in addition to an assay of direct binding. In general, such assays are used to determine the ability of a test compound to modulate or inhibit the FlgK, RpoN, or FliA gene product, e.g., flgK protein, rpoN protein, or fliA protein, or modulate or inhibit expression of the FlgK, RpoN, or FliA gene.

In general, the ability of a test compound to bind to flgK protein, rpoN protein, or fliA protein, or to the FlgK, RpoN, or FliA gene and inhibit bacterial flagellar protein biosynthesis or otherwise affect bacterial flagellar protein biosynthesis is compared to a control in which bacterial flagellar protein biosynthesis is determined in the absence of the test compound. In some cases, a predetermined reference value is used. Such reference values can be determined relative to controls, in which case a test sample that is different from the reference would indicate that the compound binds to the molecule of interest (e.g., flgK protein, rpoN protein, or fliA protein) or modulates expression (e.g., modulates or inhibits bacterial flagellar protein biosynthesis in response to Gram negative bacterial infection). A reference value can also reflect the amount of binding of an antibody or inhibitor compound to flgK protein, rpoN protein, or fliA protein compared with a standard (e.g., the affinity of antibody for flgK protein, rpoN protein, or fliA protein). In this case, a test compound that is similar to (e.g., equal to or less than) the reference would indicate that compound is a candidate compound (e.g., binds to flgK protein, rpoN protein, or fliA protein to a degree equal to or greater than a reference antibody).

This invention further pertains to novel agents identified by the above-described screening assays and uses thereof for treatments as described herein.

In one aspect the invention provides soluble assays using flgK protein, rpoN protein, or fliA protein, or a cell or tissue expressing FlgK, RpoN, or FliA gene product, either naturally occurring or recombinant. In another aspect, the invention provides solid phase based in vitro assays in a high throughput format, where flgK protein, rpoN protein, or fliA protein or its ligand is attached to a solid phase substrate via covalent or non-covalent interactions. Any one of the assays described herein can be adapted for high throughput screening.

In the high throughput assays of the invention, either soluble or solid state, it is possible to screen up to several thousand different modulators or ligands in a single day. This methodology can be used for flgK protein, rpoN protein, or fliA protein in vitro, or for cell-based or membrane-based assays comprising flgK protein, rpoN protein, or fliA protein. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin). Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I, 1993. Similarly, toxins and venoms, viral epitopes, hormones (e.g., opiates, steroids), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, polyethylene glycol linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85: 2149-2154, 1963 (describing solid phase synthesis of, e.g., peptides); Geysen et al., J. Immun. Meth. 102: 259-274, 1987 (describing synthesis of solid phase components on pins); Frank & Doring, Tetrahedron 44: 6031-6040, 1988 (describing synthesis of various peptide sequences on cellulose disks); Fodor et al., Science 251: 767-777, 1991; Sheldon et al., Clinical Chemistry 39: 718-719, 1993; and Kozal et al., Nature Medicine 2: 753-759, 1996 (all describing arrays of biopolymers fixed to solid substrates). Non-chemical approaches for fixing tag binders to substrates include other common methods, such as heat, cross-linking by UV radiation, and the like.

### Modulating or Inhibiting Expression of Polypeptides and Transcripts

The invention further provides for nucleic acids complementary to (e.g., antisense sequences to) the nucleic acid sequences of the invention. Antisense sequences are capable of modulating or inhibiting the transport, splicing or transcription of protein-encoding genes, e.g., FlgK, RpoN, or FliA-encoding nucleic acids. The modulation or inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides which are able to either bind gene or message, in either case preventing or inhibiting the production or function of the protein. The association can be through sequence specific hybridization. Another useful class of inhibitors includes oligonucleotides which cause inactivation or cleavage of protein message. The oligonucleotide can have enzyme activity which causes such cleavage, such as ribozymes. The oligonucleotide can be chemically modified or conjugated to an enzyme or composition capable of cleaving the complementary nucleic acid. One can screen a pool of many different such oligonucleotides for those with the desired activity.

General methods of using antisense, ribozyme technology and RNAi technology, to control gene expression, or of gene therapy methods for expression of an exogenous gene in this manner are well known in the art. Each of these methods utilizes a system, such as a vector, encoding either an antisense or ribozyme transcript of a phosphatase polypeptide of the invention. The term “RNAi” stands for RNA interference. This term is understood in the art to encompass technology using RNA molecules that can silence genes. See, for example, McManus, et al. Nature Reviews Genetics 3: 737, 2002. In this application, the term “RNAi” encompasses molecules such as short interfering RNA (siRNA), microRNAs (mRNA), small temporal RNA (stRNA). Generally speaking, RNA interference results from the interaction of double-stranded RNA with genes.

A. Antisense Oligonucleotides

The invention provides antisense oligonucleotides capable of binding FlgK, RpoN, or FliA messenger RNA which can inhibit polypeptide activity by targeting mRNA. Strategies for designing antisense oligonucleotides are well described in the scientific and patent literature, and the skilled artisan can design such oligonucleotides using the novel reagents of the invention. For example, gene walking/RNA mapping protocols to screen for effective antisense oligonucleotides are well known in the art, see, e.g., Ho, Methods Enzymol. 314: 168-183, 2000, describing an RNA mapping assay, which is based on standard molecular techniques to provide an easy and reliable method for potent antisense sequence selection. See also Smith, Eur. J. Pharm. Sci. 11: 191-198, 2000.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata, Toxicol Appl Pharmacol. 144: 189-197, 1997; Antisense Therapeutics, ed. Agrawal, Humana Press, Totowa, N.J., 1996. Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense polypeptides sequences of the invention (see, e.g., Gold, J. of Biol. Chem. 270: 13581-13584, 1995).

B. siRNA

“Small interfering RNA” (siRNA) refers to double-stranded RNA molecules from about 10 to about 30 nucleotides long that are named for their ability to specifically interfere with protein expression through RNA interference (RNAi). Preferably, siRNA molecules are 12-28 nucleotides long, more preferably 15-25 nucleotides long, still more. Preferably 19-23 nucleotides long and most preferably 21-23 nucleotides long. Therefore, preferred siRNA molecules are 12, 13, 14, 15, 16, 17, 18, 19, 20, 21, 22, 23, 24, 25, 26, 27 28 or 29 nucleotides in length.

RNAi is a two-step mechanism. Elbashir et al., Genes Dev., 15: 188-200, 2001. First, long dsRNAs are cleaved by an enzyme known as Dicer in 21-23 ribonucleotide (nt) fragments, called small interfering RNAs (siRNAs). Then, siRNAs associate with a ribonuclease complex (termed RISC for RNA Induced Silencing Complex) which target this complex to complementary mRNAs. RISC then cleaves the targeted mRNAs opposite the complementary siRNA, which makes the mRNA susceptible to other RNA degradation pathways.

siRNAs of the present invention are designed to interact with a target ribonucleotide sequence, meaning they complement a target sequence sufficiently to bind to the target sequence. The present invention also includes siRNA molecules that have been chemically modified to confer increased stability against nuclease degradation, but retain the ability to bind to target nucleic acids that can be present.

C. Inhibitory Ribozymes

The invention provides ribozymes capable of binding message which can inhibit polypeptide activity by targeting mRNA, e.g., inhibition of proteins involved in bacterial flagellar protein biosynthesis, e.g., flgK protein, rpoN protein, or fliA protein. Strategies for designing ribozymes and selecting the protein-specific antisense sequence for targeting are well described in the scientific and patent literature, and the skilled artisan can design such ribozymes using the novel reagents of the invention.

Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it is typically released from that RNA and so can bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The enzymatic ribozyme RNA molecule can be formed in a hammerhead motif, but can also be formed in the motif of a hairpin, hepatitis delta virus, group I intron or RnaseP-like RNA (in association with an RNA guide sequence). Examples of such hammerhead motifs are described by Rossi, Aids Research and Human Retroviruses 8: 183, 1992; hairpin motifs by Hampel, Biochemistry 28: 4929, 1989, and Hampel, Nuc. Acids Res. 18: 299, 1990; the hepatitis delta virus motif by Perrotta, Biochemistry 31: 16, 1992; the RnaseP motif by Guerrier-Takada, Cell 35: 849, 1983; and the group I intron by Cech U.S. Pat. No. 4,987,071. The recitation of these specific motifs is not intended to be limiting; those skilled in the art will recognize that an enzymatic RNA molecule of this invention has a specific substrate binding site complementary to one or more of the target gene RNA regions, and has nucleotide sequence within or surrounding that substrate binding site which imparts an RNA cleaving activity to the molecule.

### Methods of Treatment

Also described herein are both prophylactic and therapeutic methods of treating a subject at risk of (or susceptible to) a disorder or a method of preventing or treating a Gram negative bacterial infection, e.g., Campylobacter jejuni infection by administering an antagonist of bacterial flagellar protein biosynthesis.

### Prophylactic Methods

An aspect of the invention relates to methods for preventing or treating in a subject a Gram negative bacterial infection by administering vaccine composition comprising an effective immunizing amount of a bacterial flagellar protein or a protein involved in bacterial flagellar biosynthesis and a pharmaceutically acceptable carrier, wherein said vaccine composition is effective in a vertebrate subject to reduce or eliminate Gram-negative bacterial infection. The therapeutic composition can also be an antagonist of bacterial flagellar protein biosynthesis administered to the vertebrate subject in an amount effective to reduce or eliminate the bacterial infection. Subjects at risk for a disorder or undesirable symptoms that are caused or contributed to by Gram negative bacterial infection can be identified by, for example, any of a combination of diagnostic or prognostic assays as described herein or are known in the art. In general, such disorders involve undesirable activation of the innate immune system, e.g., as a result of Gram negative bacterial infection. Administration of the agent as a prophylactic agent can occur prior to the manifestation of symptoms, such that the symptoms are prevented, delayed, or diminished compared to symptoms in the absence of the agent. In some aspects, the agent decreases bacterial flagellar protein biosynthesis. In some aspects, the agent decreases synthesis or activity of proteins, e.g., flgK protein, rpoN protein, or fliA protein, involved in bacterial flagellar protein biosynthesis. The appropriate agent can be identified based on screening assays described herein. In general, such agents specifically bind to flgK polypeptide, rpoN polypeptide, or fliA polypeptide.

### Therapeutic Methods

An aspect of the invention relates to methods for preventing or treating in a subject a Gram negative bacterial infection by administering vaccine composition comprising an effective immunizing amount of a bacterial flagellar protein or a protein involved in bacterial flagellar biosynthesis and a pharmaceutically acceptable carrier, wherein said vaccine composition is effective in a vertebrate subject to reduce or eliminate Gram-negative bacterial infection. A further aspect pertains to methods of modulating or inhibiting FlgK, RpoN, or FliA gene expression or flgK polypeptide, rpoN polypeptide, or fliA polypeptide activity related to Gram negative bacterial flagellar protein biosynthesis which can result in inhibition of Gram negative bacterial infection for therapeutic purposes. The method can include contacting a cell with an agent that modulates or inhibits one or more activities of FlgK, RpoN, or FliA gene expression, or inhibition of protein activity, e.g., flgK protein, rpoN protein, or fliA protein, involved in bacterial flagellar protein biosynthesis. The agent can be a compound that specifically binds to flgK protein, rpoN protein, or fliA protein to treat or prevent Gram negative bacterial infection. The agent can be an antibody or a protein, a naturally-occurring cognate ligand of a FlgK, RpoN, or FliA protein, a peptide, a FlgK, RpoN, or FliA protein peptidomimetic, a small non-nucleic acid organic molecule, or a small inorganic molecule. These modulatory methods can be performed in vitro (e.g., by culturing the cell with the agent) or, alternatively, in vivo (e.g., by administering the agent to a subject).

The present invention provides methods for preventing or treating an individual affected by a disease or disorder, e.g., Gram negative bacterial infection. In one aspect, the method involves administering a vaccine composition comprising an effective immunizing amount of a bacterial flagellar protein or a protein involved in bacterial flagellar biosynthesis and a pharmaceutically acceptable carrier, or a vaccine composition comprising a therapeutic agent such as an inhibitor of proteins, e.g., flgK protein, rpoN protein, or fliA protein, involved in bacterial flagellar protein biosynthesis. Conditions that can be treated by agents include those in which a subject is treated for Gram negative bacterial infection, e.g., Campylobacter jejuni infection.

Other disorders that can be treated by the new methods and compositions include Gram negative bacterial infection including but not limited to, Campylobacter jejuni infection, Enterobacteriacea infection, including but not limited to, Escherichia, Shigella, Edwardsiella, Salmonella, Citrobacter, Klebsiella, Enterobacter, Hafnia, Serratia, Proteus, Morganella, Providencia, Yersinia, Erwinia, Buttlauxella, Cedecea, Ewingella, Kluyvera, Tatumella and Rahnella. Other exemplary Gram-negative organisms not in the family Enterobacteriacea include, but are not limited to, Pseudomonas aeruginosa, Stenotrophomonas maltophilia, Burkholderia, Cepacia, Gardenerella, Vaginalis, and Acinetobacter species.

Successful treatment of disorders related to Gram negative bacterial infection can be brought about by techniques that serve to inhibit proteins, e.g., flgK protein, rpoN protein, or fliA protein, involved in bacterial flagellar protein biosynthesis. For example, compounds, e.g., an agent identified using an assay described herein, such as an antibody, that prove to exhibit negative modulatory activity, can be used to prevent and/or ameliorate symptoms of Gram negative bacterial infection. Such molecules can include, but are not limited to peptides, phosphopeptides, small organic or inorganic molecules, or antibodies (including, for example, polyclonal, monoclonal, humanized, anti-idiotypic, chimeric or single chain antibodies, and F_{ab}, F(_{ab}′)₂ and F_{ab} expression library fragments, scFV molecules, and epitope-binding fragments thereof). In particular, antibodies and derivatives thereof (e.g., antigen-binding fragments thereof) that specifically bind to proteins, e.g., flgK protein, rpoN protein, or fliA protein, involved in bacterial flagellar protein biosynthesis in a cell.

### Kits

The invention provides kits comprising the compositions, e.g., nucleic acids, expression cassettes, vectors, cells, polypeptides (e.g., flgK polypeptide, rpoN polypeptide, or fliA polypeptide) and/or antibodies of the invention. The kits also can contain instructional material teaching the methodologies and uses of the invention, as described herein.

### Therapeutic Applications

The compounds and modulators identified by the methods of the present invention can be used in a variety of methods of treatment. Thus, the present invention provides compositions and methods for treating Gram negative bacterial infection.

Exemplary infectious disease, include but are not limited to, bacterial diseases. The polypeptide or polynucleotide of the present invention can be used to treat or detect infectious agents. For example, by increasing the immune response, particularly increasing the proliferation and differentiation of B and/or T cells, infectious diseases can be treated. The immune response can be increased by either enhancing an existing immune response, or by initiating a new immune response. Alternatively, the polypeptide or polynucleotide of the present invention can also directly inhibit the infectious agent, without necessarily eliciting an immune response.

Exemplary infectious disease, include but are not limited to, Gram negative infections. Gram-negative bacterial agents that can cause disease or symptoms and that can be treated or detected by a polynucleotide or polypeptide of the present invention include, but not limited to, the following Gram-negative bacterial families. Bacteremia can be caused by Gram-negative bacteria. Gram-negative bacteria have thin walled cell membranes consisting of a single layer of peptidoglycan and an outer layer of lipopolysacchacide, lipoprotein, and phospholipid. Exemplary Gram-negative organisms include, but are not limited to, Enterobacteriacea consisting of Campylobacter, Escherichia, Shigella, Edwardsiella, Salmonella, Citrobacter, Klebsiella, Enterobacter, Hafnia, Serratia, Proteus, Morganella, Providencia, Yersinia, Erwinia, Buttlauxella, Cedecea, Ewingella, Kluyvera, Tatumella and Rahnella. Other exemplary Gram-negative organisms not in the family Enterobacteriacea include, but are not limited to, Pseudomonas aeruginosa, Stenotrophomonas maltophilia, Burkholderia, Cepacia, Gardenerella, Vaginalis, and Acinetobacter species.

Preferably, treatment using vaccine composition or a polypeptide or polynucleotide of the present invention could either be by administering an effective amount of a polypeptide to the mammalian or avian patient having a Gram negative bacterial infection or at risk for a Gram negative bacterial infection. Moreover, the polypeptide or polynucleotide of the present invention can be used as an antigen in a vaccine to raise an immune response against Gram negative bacterial infectious disease.

### Formulation and Administration of Pharmaceutical Compositions

An aspect of the invention provides vaccine composition comprising an effective immunizing amount of a bacterial flagellar protein or a protein involved in bacterial flagellar biosynthesis and a pharmaceutically acceptable carrier, wherein said vaccine composition is effective in a vertebrate subject to reduce or eliminate Gram-negative bacterial infection. A further aspect provides pharmaceutical compositions comprising nucleic acids, polypeptides (including antibodies), peptidomimetic, small non-nucleic acid organic molecule, or small inorganic molecule of the invention. As discussed above, the nucleic acids, polypeptides, small chemical molecule of the invention can be used to inhibit expression of an endogenous flgK polypeptide, rpoN polypeptide, or fliA polypeptide. Such inhibition in a cell or a non-human animal can generate a screening modality for identifying compounds to treat or ameliorate a Gram negative bacterial infection. Administration of a pharmaceutical composition of the invention to a subject is used to generate a toleragenic immunological environment in the subject. This can be used to tolerize the subject to an antigen.

The vaccine compositions, or nucleic acids, polypeptides, or small chemical molecule can be combined with a pharmaceutically acceptable carrier (excipient) to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are described in detail in the scientific and patent literature, see e.g., the latest edition of Remington's Pharmaceutical Science, Mack Publishing Company, Easton, Pa. (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the peptide or polypeptide of the invention and on its particular physio-chemical characteristics.

In one aspect, a solution of the vaccine composition or nucleic acids, peptides or polypeptides are dissolved in a pharmaceutically acceptable carrier, e.g., an aqueous carrier if the composition is water-soluble. Examples of aqueous solutions that can be used in formulations for enteral, parenteral or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of peptide in these formulations can vary widely, and will be selected primarily based on fluid volumes, viscosities, body weight and the like in accordance with the particular mode of administration selected and the patient's needs.

Solid formulations can be used for enteral (oral) administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral administration. The carrier can be selected from various oils including those of petroleum, animal, vegetable or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Vaccine compositions or nucleic acids, polypeptides, or small chemical molecules, when administered orally, can be protected from digestion. This can be accomplished either by complexing the nucleic acid, peptide or polypeptide with a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g., Fix, Pharm Res. 13: 1760-1764, 1996; Samanen, J. Pharm. Pharmacol. 48: 119-135, 1996; U.S. Pat. No. 5,391,377, describing lipid compositions for oral delivery of therapeutic agents (liposomal delivery is discussed in further detail, infra). 26728-14571

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. See, e.g., Sayani, Crit. Rev. Ther. Drug Carrier Syst. 13: 85-184, 1996. For topical, transdermal administration, the agents are formulated into ointments, creams, salves, powders and gels. Transdermal delivery systems can also include, e.g., patches.

Vaccine compositions or nucleic acids, polypeptides, or small chemical molecule as aspects of the invention can also be administered in sustained delivery or sustained release mechanisms, which can deliver the formulation internally. For example, biodegradeable microspheres or capsules or other biodegradeable polymer configurations capable of sustained delivery of a peptide can be included in the formulations of the invention (see, e.g., Putney, Nat. Biotechnol. 16: 153-157, 1998).

For inhalation, vaccine compositions or nucleic acids, nucleic acids, polypeptides, or small chemical molecule as aspects of the invention can be delivered using any system known in the art, including dry powder aerosols, liquids delivery systems, air jet nebulizers, propellant systems, and the like. See, e.g., Patton, Biotechniques 16: 141-143, 1998; product and inhalation delivery systems for polypeptide macromolecules by, e.g., Dura Pharmaceuticals (San Diego, Calif.), Aradigrn (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

Avian subjects can be administered the vaccine compositions or nucleic acids, polypeptides, or small chemical molecule as aspects of the present invention by any suitable means. Exemplary means are oral administration (e.g., in the feed or drinking water), intramuscular injection, subcutaneous injection, intravenous injection, intra-abdominal injection, eye drop, or nasal spray. Avian subjects can also be administered the compounds in a spray cabinet, i.e., a cabinet in which the birds are placed and exposed to a vapor containing vaccine, or by coarse spray. When administering the compounds described herein to birds post-hatch, administration by subcutaneous injection or spray cabinet are commonly used techniques.

The vaccine compositions or nucleic acids, polypeptides, or small chemical molecule as aspects of the present invention can also be administered in ovo. The in ovo administration of the compounds involves the administration of the compounds to the avian embryo while contained in the egg. The compounds can be administered to any suitable compartment of the egg (e.g., allantois, yolk sac, amnion, air cell, or into the avian embryo itself), as would be apparent to one skilled in the art. Eggs administered the compounds can be fertile eggs which are preferably in the last half, and more preferably the last quarter, of incubation. Chicken eggs are preferably treated on about day 18 of incubation, although other time periods can be employed. Those skilled in the art will appreciate that the present invention can be carried out at various predetermined times in ovo.

Eggs can be administered the vaccine compositions or nucleic acids, polypeptides, or small chemical molecule of the invention by any means which transports the compound through the shell. A common method of administration is, however, by injection. For example, the compound can injected into an extraembryonic compartment of the egg (e.g., yolk sac, amnion, allantois, air cell) or into the embryo itself. As an example, the site of injection can be within the region defined by the amnion, including the amniotic fluid and the embryo itself. By the beginning of the fourth quarter of incubation, the amnion is sufficiently enlarged that penetration thereof is assured nearly all of the time when the injection is made from the center of the large end of the egg along the longitudinal axis.

The mechanism of egg injection is not critical, but it is preferred that the method not unduly damage the tissues and organs of the embryo or the extraembryonic membranes surrounding it so that the treatment will not decrease hatch rate. The size of the needle and the length of penetration can be determined by one skilled in the art. A pilot hole can be punched or drilled through the shell prior to insertion of the needle to prevent damaging or dulling of the needle. If desired, the egg can be sealed with a substantially bacteria-impermeable sealing material such as wax or the like to prevent subsequent entry of undesirable bacteria.

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's, Chapters 37-39.

Vaccine compositions or nucleic acids, polypeptides, or small chemical molecule of the invention can be delivered alone or as pharmaceutical compositions by any means known in the art, e.g., systemically, regionally, or locally (e.g., directly into, or directed to, a tumor); by intraarterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa). Actual methods for preparing administrable compositions will be known or apparent to those skilled in the art and are described in detail in the scientific and patent literature, see e.g., Remington's. For a “regional effect,” e.g., to focus on a specific organ, one mode of administration includes intra-arterial or intrathecal (IT) injections, e.g., to focus on a specific organ, e.g., brain and CNS (see e.g., Gurun, Anesth Analg. 85: 317-323, 1997). For example, intra-carotid artery injection if preferred where it is desired to deliver a nucleic acid, peptide or polypeptide of the invention directly to the brain. Parenteral administration is a preferred route of delivery if a high systemic dosage is needed. Actual methods for preparing parenterally administrable compositions will be known or apparent to those skilled in the art and are described in detail, in e.g., Remington's, See also, Bai, J. Neuroimmunol. 80: 65-75, 1997; Warren, J. Neurol. Sci. 152: 31-38, 1997; Tonegawa, J. Exp. Med. 186:507-515, 1997.

In one aspect, the pharmaceutical formulations comprising vaccine compositions or nucleic acids, polypeptides, or small chemical molecule of the invention are incorporated in lipid monolayers or bilayers, e.g., liposomes, see, e.g., U.S. Pat. Nos. 6,110,490; 6,096,716; 5,283,185; 5,279,833. Aspects of the invention also provide formulations in which water soluble nucleic acids, peptides or polypeptides of the invention have been attached to the surface of the monolayer or bilayer. For example, peptides can be attached to hydrazide-PEG-(distearoylphosphatidyl)ethanolamine-containing liposomes (see, e.g., Zalipsky, Bioconjug. Chem. 6:705-708, 1995). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla, J. Pharm. Sci. 85: 5-8, 1996), transmucosally, or orally. The invention also provides pharmaceutical preparations in which the nucleic acid, peptides and/or polypeptides of the invention are incorporated within micelles and/or liposomes (see, e.g., Suntres, J. Pharm. Pharmacol. 46: 23-28, 1994; Woodle, Pharm. Res. 9: 260-265, 1992). Liposomes and liposomal formulations can be prepared according to standard methods and are also well known in the art, see, e.g., Remington's; Akimaru, Cytokines Mol. Ther. 1: 197-210, 1995; Alving, Immunol. Rev. 145: 5-31, 1995; Szoka, Ann. Rev. Biophys. Bioeng. 9: 467, 1980, U.S. Pat. Nos. 4,235,871, 4,501,728 and 4,837,028.

In one aspect, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models, e.g., of inflammation or disorders involving undesirable inflammation, to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography, generally of a labeled agent. Animal models useful in studies, e.g., preclinical protocols, are known in the art, for example, animal models for inflammatory disorders such as those described in Sonderstrup (Springer, Sem Immunopathol. 25: 35-45, 2003) and Nikula et al., Inhal. Toxicol. 4(12): 123-53, 2000), and those known in the art, e.g., for Gram-negative bacterial infection, e.g., Campylobacter jejuni infection.

As defined herein, a therapeutically effective amount of vaccine compositions, protein or polypeptide such as an antibody (i.e., an effective dosage) ranges from about 0.001 to 30 mg/kg body weight, for example, about 0.01 to 25 mg/kg body weight, about 0.1 to 20 mg/kg body weight, or about 1 to 10 mg/kg, 2 to 9 mg/kg, 3 to 8 mg/kg, 4 to 7 mg/kg, or 5 to 6 mg/kg body weight. The protein or polypeptide can be administered one or several times per day or per week for between about 1 to 10 weeks, for example, between 2 to 8 weeks, between about 3 to 7 weeks, or about 4, 5, or 6 weeks. In some instances the dosage can be required over several months or more. The skilled artisan will appreciate that certain factors can influence the dosage and timing required to effectively treat a subject, including, but not limited to the severity of the disease or disorder, previous treatments, the general health and/or age of the subject, and other diseases present. Moreover, treatment of a subject with a therapeutically effective amount of an agent such as a protein or polypeptide (including an antibody) can include a single treatment or, preferably, can include a series of treatments.

For antibodies, the dosage is generally 0.1 mg/kg of body weight (for example, 10 mg/kg to 20 mg/kg). Partially human antibodies and fully human antibodies generally have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al, J. Acquired Immune Deficiency Syndromes and Human Retrovirology, 14: 193, 1997).

Aspects of present invention encompass vaccine composition comprising an effective immunizing amount of a bacterial flagellar protein or a protein involved in bacterial flagellar biosynthesis and a pharmaceutically acceptable carrier, wherein said vaccine composition is effective in a vertebrate subject to reduce or eliminate Gram-negative bacterial infection, or agents or compounds that modulate expression or activity of FlgK, RpoN, or FliA gene expression or flgK protein, rpoN protein, or fliA protein to modulate or inhibit bacterial flagellar protein biosynthesis of a Gram negative bacteria. The agents or compounds are useful in a method for prevention or treatment of Gram negative bacterial infection. An agent can be, for example, a small chemical molecule. Such small chemical molecules include, but are not limited to, peptides, peptidomimetics (e.g., peptoids), amino acids, amino acid analogs, small non-nucleic acid organic compounds or inorganic compounds (I.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small chemical molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small chemical molecule depend upon the potency of the small chemical molecule with respect to the expression or activity to be modulated. When one or more of these small chemical molecules is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody or fragment thereof can be linked, e.g., covalently and/or with a linker to another therapeutic moiety such as a therapeutic agent or a radioactive metal ion, to form a conjugate. Therapeutic agents include, but are not limited to, antibiotics (e.g., dactinomycin (formerly actinomycin), bleomycin, mithramycin, and anthramycin (AMC)).

The conjugates described herein can be used for modifying a given biological response. For example, the moiety bound to the antibody can be a protein or polypeptide possessing a desired biological activity. Such proteins can include, for example, a toxin such as abrin, ricin A, Pseudomonas exotoxin, or diphtheria toxin; a protein such as tumor necrosis factor, α-interferon, β-interferon, nerve growth factor, platelet derived growth factor, tissue plasminogen activator; or, biological response modifiers.

Alternatively, an antibody can be conjugated to a second antibody to form an antibody heteroconjugate as described by Segal in U.S. Pat. No. 4,676,980.

The pharmaceutical compositions can be included in a container, pack, or dispenser together with instructions for administration.

Compounds as described herein can be used for the preparation of a medicament for use in any of the methods of treatment described herein.

The pharmaceutical compositions are generally formulated as sterile, substantially isotonic and in full compliance with all Good Manufacturing Practice (GMP) regulations of the U.S. Food and Drug Administration.

### Treatment Regimens: Pharmacokinetics

The pharmaceutical composition aspects of the invention can be administered in a variety of unit dosage forms depending upon the method of administration. Dosages for typical vaccine compositions or nucleic acids, peptide and polypeptide pharmaceutical compositions are well known to those of skill in the art. Such dosages are typically advisory in nature and are adjusted depending on the particular therapeutic context or patient tolerance. The amount of nucleic acid, peptide or polypeptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton, Peptides 18: 1431-1439, 1997; Langer, Science 249: 1527-1533, 1990.

In therapeutic applications, compositions are administered to a patient at risk for Gram negative bacterial infection or suffering from Gram negative bacterial infection in an amount sufficient to at least partially arrest or prevent the condition or a disease and/or its complications. For example, in one aspect, vaccine composition comprising a soluble peptide pharmaceutical composition dosage for intravenous (IV) administration would be about 0.01 mg/hr to about 1.0 mg/hr administered over several hours (typically 1, 3, or 6 hours), which can be repeated for weeks with intermittent cycles. Considerably higher dosages (e.g., ranging up to about 10 mg/ml) can be used, particularly when the drug is administered to a secluded site and not into the blood stream, such as into a body cavity or into a lumen of an organ, e.g., the cerebrospinal fluid (CSF).

The following examples of specific aspects for carrying out the present invention are offered for illustrative purposes only, and are not intended to limit the scope of the present invention in any way.

### EXEMPLARY ASPECTS

### Example 1

### Construction and Phenotypic Analysis of rpoN, fliA and flgk Mutants

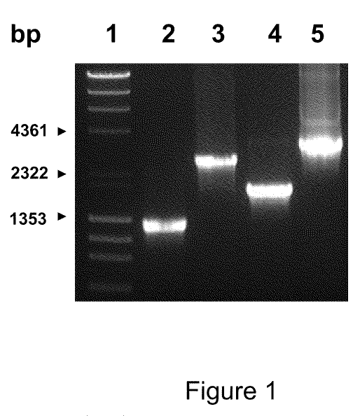

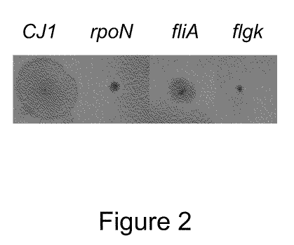

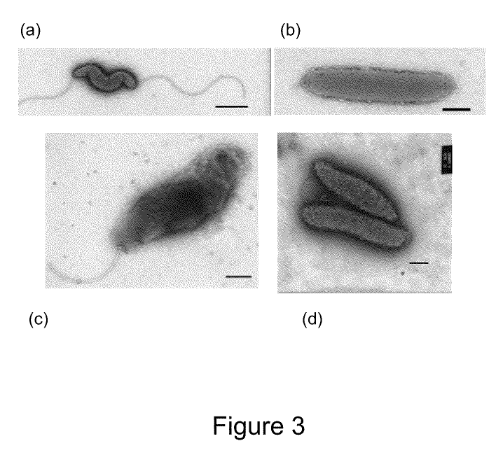

To investigate the role of the alternative sigma factor σ⁵⁴ in the regulation of C. jejuni colonization, the rpoN gene was inactivated by allelic exchange with a defective copy of the gene carrying a kanamycin-resistance cassette. In addition, to test the role of the hook filament junction protein, FlgK, in the assembly of the C. jejuni flagellum and to study its impact on colonization, the flgk gene was also inactivated as described above. Correct replacement of the wild type rpoN and flgk genes was confirmed by PCR analysis (FIG. 1). Phenotypic analyses of the rpoN, flgk and fliA (Carrillo et al, J Biol Chem 279:20327-20338, 2004) mutants demonstrated that rpoN and flgk were non motile with 90% reduction in motility compared to the wild type CJ1 in a soft agar motility assay, while fliA mutant showed 75% reduction in motility compared to the wild type (FIG. 2). Electron microscopy revealed that the rpoN mutant was aflagellate and the cells lacked the typical spiral morphology, while the flgk mutant produced only the hook structure. The fliA mutant possessed a truncated flagellar filament in most cells (FIG. 3).

FIG. 1 shows PCR confirmation of insertional inactivation of the rpoN and flgk mutants of C. jejuni NCTC11168. Lanes: 1—DRIgest III DNA marker; 2- and 3-, rpoN PCR amplification products of the wild-type and mutant, respectively; 4- and 5-, flgk PCR amplification products of the wild-type and mutant, respectively.

FIG. 2 shows motility of the C. jejuni wild-type (CJ1) and mutants after incubation for 48 hr at 37° C. on 0.4% MH agar.

FIG. 3 shows electron micrographs of wild-type C. jejuni (CJ1) and the mutant strains. (a) flagellated wild-type CJ1 (bar, 1 μm) (b) fgk mutant produced hook structures visible at both poles (bar, 0.25 μm) (c) fliA with a truncated flagellum (bar, 0.25 μm). (d) aflagellated rpoN (bar, 0.25 μm).

### Example 2

### C. jejuni Protein Secretion

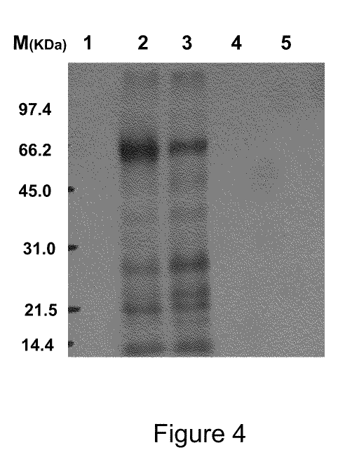

Secretion assays were performed in the presence and absence of FBS to determine whether the C. jejuni mutants were capable of secreting Cia proteins. FBS serves as an artificial signal to stimulate the synthesis and secretion of the Cia proteins. The C. jejuni strains were incubated in EMEM supplemented with FBS and the secretion of proteins was determined by [³⁵S]-methionine labelling coupled with autoradiography. The Cia proteins were detected in the supernatant fluids of the wild-type and fliA mutant only (FIG. 4). No secreted proteins were detected in the supernatant fluids of either the rpoN or the fgk mutant. The Cia proteins were not detected in the supernatant fluids of wild-type when FBS was omitted from the incubating medium.

To determine if the ciaB gene was expressed in the rpoN mutant, reverse transcription-PCR analysis was performed. A band representing the ciaB transcript was clearly evident in this mutant (data not shown), demonstrating that ciaB expression is independent of the alternative sigma factor σhu 54.

FIG. 4 shows secretion of Cia proteins by C. jejuni wild type (CJ1),fliA, rpoN and flgk mutants. C. jejuni cells were grown on Mueller Hinton blood plates and labelled in minimum essential medium in the presence (Lanes 2-5) and absence of fetal bovine serum (Lane 1). Supernatants were analyzed by SDS-polyacrylamide gel electrophoresis and autoradiography, as outlined in Materials and methods. Lanes: Lanes 1&2: C. jejuni wild type (CJ1), Lane 3: fliA mutant, Lane 4: flgk mutant, Lane 5: rpoN mutant,

### Example 3

### In Vitro Infection by rpoN, fliA and flgk Mutants

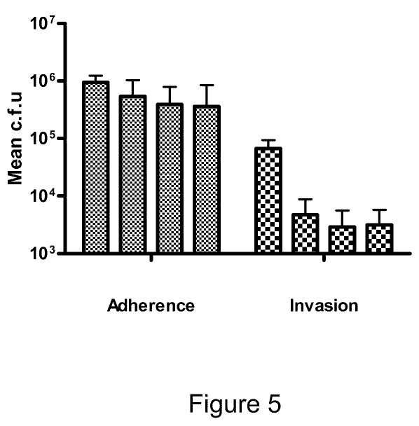

To examine the role of the rpoN and fl/A genes in regulating C. jejuni infection, we tested the in vitro adhesion and invasion ability of the rpoN and fliA mutants and compared their infective ability with the structural mutant flgk. HeLa cells were infected with the wild-type C. jejuni and the mutants and the total number of bacteria that had adhered and invaded was determined. Only rpoN and fgk mutants showed significantly less (P<0.01) adherence compared to the wild type C. jejuni (FIG. 5). A significant decrease, amounting to a 10-fold reduction, was noted in the number of bacteria internalized for the C. jejuni fliA, rpoN and flgk mutants compared to the C. jejuni wild-type strain (P<0.001). The fliA mutant, which expresses a truncated flagellum and secretes Cia proteins, did not show any significant difference (P>0.05) in internalization compared to the rpoN or flgk mutants.

FIG. 5 shows adherence to and invasion of Hela cells with wild-type C. jejuni (CJ1), fliA, rpoN and flgk mutants. The adherence and invasion data are averages of three independent experiments performed in duplicate. As detected by the Kiruskal-Wallis test the adherence and invasion value of each mutant was significantly different from the wild-type CJ 1, except for fliA mutant.

### Example 4

### Colonization of Chicks by C. jejuni rpoN, fliA and flgk Mutants

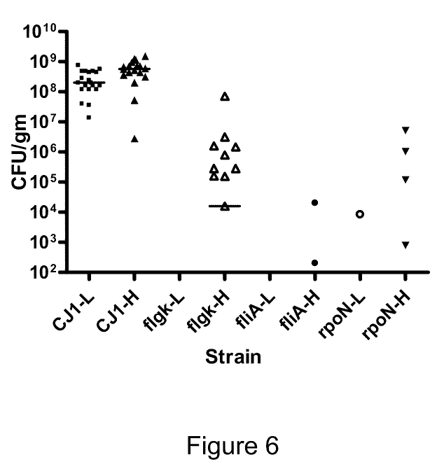

To determine the colonization capacity of C. jejuni rpoN, fliA andfigk insertional mutants defective for motility and secretion, we infected day old chicks with two different doses, 5×10⁷ and 5×10⁸ cfu. In addition, 15 uninfected chicks were comingled with five infected chicks that had each been given 5×10⁸ cfu of one of the variants in order to measure horizontal transmission. The wild type C. jejuni rapidly spread to the uninfected contact birds, and by day 7 all birds in the group were colonized with a median of 10⁸ cfu per gram of cecal contents. In contrast, no horizontal transmission was detected for any of the mutants. In the groups where all birds were challenged with 5×10⁷ cfu, none of the mutants were able to colonize any of the 20 birds with the exception of one bird infected by the rpoN mutant. At a challenge dose of 5×10⁸ cfu, only the flgk mutant colonized at a median of 8×10³ cfu/gm, while the wild-type strain colonized all birds with a median recovery of 108 cfu/gm and 10⁹ cfu/gm at the lower and higher infective doses respectively. (FIG. 6)

FIG. 6 shows caecal colonization levels (cfu/gm) of wild-type C. jejuni (CJ1), fliA, rpoN and flgk mutants. Twenty birds were orally infected with two different doses (5×10⁷-L and 5×10⁸-H) of each strain at one day of hatch. The median values for each group are indicated by horizontal lines. The Kruskal-Wallis test revealed that the median cfu/gm was significantly different for all groups (P<0.0001). Tukey's pairwise comparisons for colonization levels between the two doses for each strain showed a significant difference (P<0.001) only for CJ1-L and CJ1-H. In the day-old chicken colonization model used, a bacterial load of 4×10² is the minimum detectable

### Example 5

### Therapeutic Applications of Inhibitors of Flagellar Protein Biosynthesis in Gram Negative Bacteria

Flagellar-based motility has been shown to be a requirement for the virulence of many enteric bacterial pathogens including C. jejuni. Biosynthesis of flagella requires the involvement of more than 40 structural and regulatory proteins, including a type III secretion system necessary for flagellar assembly. Recent studies (Hendrixson and DiRita, Mol Microbiol 50:687-702, 2003; Hendrixson and DiRita, Mol Microbiol 52:471-484, 2004; Carrillo et al., J Biol Chem 279:20327-20338, 2004) have shown that the flagellar sigma factor σ²⁸ (/iA) and the alternative sigma factor σ⁵⁴ (rpoM) regulate a large number of genes responsible for the expression and function of the C. jejuni flagella and are required for pathogenicity and intestinal colonization. In the present study we have investigated the multiple roles of the C. jejuni flagellum: motility, protein secretion and invasion of host cells. We have also used a flagellar structural mutant, flgk, and compared its colonization potential with the above regulatory mutants in order to examine any indirect effects mediated by σ²⁸ and σ⁵⁴ genes in establishing C. jejuni colonization.

Previous studies have shown that C. jejuni secretes Cia proteins upon co-cultivation with intestinal epithelial cells (Konkel et al., Mol Microbiol 32:691-701, 1999), and it appears to use the flagellar structure as the export apparatus for their secretion. Additionally it was shown that the minimum requirement for secretion is a functional basal body, hook and at least one of the filament proteins (Konkel et al., Mol Microbiol 32:691-701, 1999). Our results showed that the C. jejuni fliA mutant is capable of Cia protein secretion, but that both rpoN and flgk mutants were unable to secrete Cia proteins. Our results are in agreement with those of Konkel et al. (2004) and reiterate that fliA is not responsible for transcription of the genes encoding the flagellar export apparatus in C. jejuni. As demonstrated by RT-PCR analysis, the ciaB gene is transcribed in the rpoN mutant and it appears that the inability to secrete C₁₋a proteins is due to the non-functional flagellar secretory apparatus in this mutant. This result supports the data of Hendrixson and DiRita (2003) who showed that transcription of the genes associated with the formation of the flagellar secretory apparatus in C. jejuni. is σ⁵⁴-dependant but not σ²⁸-dependant. The sequence of the NCTC11168 ciaB gene shows the presence of a potential σ⁷⁰ promoter sequence with a typical −10 consensus sequence (TATAAT) present 9 bp upstream from the ribosomal binding site of the gene. Even though the ciaB gene does not show a typical −35 consensus sequence for the σ⁷⁰ promoter, it seems to possess an extended TG motif with the −10 sequence as shown to be present in E. coli which can compensate for a −35 consensus sequence (Kumar et al, J Mol Biol 232:406-418, 1993).

In the flagellar biosynthesis pathway of enteric bacteria, FlgK and FkgL are the two hook-associated proteins, and in Salmonella typhimurium it has been shown that flgk mutants retain FlgD, the hook-capping protein, and therefore cannot assemble the filament (Oshnishi et al, 1994). In C. jejuni, the function of FlgK protein in the flagellar assembly pathway has not been investigated, and thus we investigated whether a flgk mutant could express a functional hook even though it was unable to produce a filament. Electron microscopy of the flgk mutant showed the hook structure was present but there was no filament (FIG. 3). This mutant did not secrete any Cia proteins (FIG. 4) indicating that FlgK protein can be necessary to complete the assembly of the C. jejuni flagellar secretory apparatus.

We also studied the ability of the fliA, rpoN and flgk mutants to invade HeLa cells in vitro in order to understand the role of fliA and rpoN in C. jejuni pathogenesis and colonization. The rpoN and flgk mutants showed a significant reduction in adherence to HeLa cells compared to the wild type C. jejuni but the adherence of the fliA mutant was not significantly different from the wild-type. In C. jejuni, the flagellar filament protein flagellin is encoded by flaA and flaB, and expression of the flaA gene results from σ²⁸ promoter activity while the flaB gene expression results from σ⁵⁴ promoter activity (Guerry et al., 1990). Previous studies have shown that a flaA⁻ flaB⁺ mutant of C. jejuni displays a truncated flagellar filament composed of only the flaB protein (Guerry et al., 1991). Our results show that fliA mutants display a truncated flagellar filament as previously observed by Jagannathnan et al., (2001). However, flaA⁻ flaB⁺ mutant of C. jeiuni F38011 which also expresses a truncated flagellar filament attached to INT 407 cells even better than its wild-type parent (Konkel et al., J Bacteriol 186:3296-3303, 2004) while the same mutant of C. jejuni 81116 showed no difference in adherence compared to its wild-type parent (Grant et al., Infect Immun 61:1764-1771, 1993). The adherence of the flgk mutant in the present study is consistent with the adherence levels of the flgE2 hook mutant of C. jejuni F38011 (Konkel et al., J Bacteriol 186:3296-3303, 2004). The role of the flagella in adherence to epithelial cells has been controversial. Earlier studies have implicated a role for flagellar-mediated adherence of C. jejuni to epithelial cells (McSweegan and Walker, Infect Immun 53:141-148, 1986). But the evidence shown in later work revealed that flagella can not be involved in C. jejuni adherence to epithelial cells (Grant et al., Infect Immun 61:1764-1771, 1993). However in the present study, the inability of the rpoN mutant which is aflagellate and the flgk mutant with only a hook structure to adhere to epithelial cells can indicate a role for flagellar based adherence of C. jejuni. A significant reduction was observed in the internalization of all three mutants compared to the wild type. It is also noteworthy to mention that no significant difference was observed in internalization of the fliA mutant, which is secretion positive and possess a truncated filament compared to the secretion negative, aflagellated rpoN mutant. These results indicate that both fliA and rpoN genes regulate the process of binding and internalization of C. jejuni to epithelial cells in a flagellar independent mechanism, in addition to their direct regulation of flagellar class II and III genes. Similarly it has been shown in Pseudomonas aeruginosa, that the defects associated with mutation of the rpoN gene involving virulence of this bacterium were greater than with strains that were specifically pilin negative (Kazmierczak et al., Microbiol & Mol Biology Reviews 69:527-543, 2005).

In parallel with the results of in vitro infection experiments, the colonization abilities of the fliA, rpoN and flgk mutants were tested in a day-old chicken model. Both fliA and rpoN mutants were completely attenuated for caecal colonization at both doses tested. The flgk mutant demonstrated a colonization capacity approximately 100.000-fold lower than the wild type parent at the higher infective dose of 5×10⁸. In parallel with the results of the in vitro infection, the defect in colonization capacity of the present fliA mutant is much more severe than a flaA⁻ flaB⁺ mutant of the same strain (NCTC11168) tested in our laboratory which demonstrated colonization levels almost comparable to wild type levels (Biswas et al., 13^{th} Int'l Workshop on Campylobacter, Helicobacter and Related Organisms, Gold Coast, Queensland, Australia, p. 65 (Abstract), E3, 2005). Also, based on the findings of our earlier studies that C. jejuni secretes Cia proteins in the presence of chicken serum and mucus (Biswas et al., Current Microbiology, 2006), we speculated that a fliA mutant that was competent for Cia protein secretion would have a similar advantage as flaA⁻ flaB⁺ in establishing or initiating colonization in the chicken cecum over the aflagellated, secretion-negative rpoN mutant. However this was shown to be untrue. In an attempt to identify genes involved in colonization, (Ziprin et al., Avian Diseases 45:549-557, 2001) found that a ciaB mutant of C. jejuni F38011 could colonize the chicken cecum only 20-40% as effectively as the wild-type strain, but in a separate experiment they found that the ciaB mutant colonized the crop at a level ranging from 10³ to 1 cfu/gm compared to the wild-type strain (Ziprin et al., Avian Diseases 45:549-557, 2001). We also observed that all mutants showed in vitro growth rates similar to their respective wild-type parents indicating that the colonization defects were due to specific mutations in the genes, but not due to growth defects. In the horizontal challenge model, the five birds infected with the wild-type CJ1 at dose of 5×10³ rapidly spread to the uninfected contact birds and all birds in the group were colonized with a median of 108 by day 7. In contrast, no horizontal transmission was detected in the uninfected birds for any of the mutants indicating the severity of the defects caused by fliA, rpoN and flgk gene mutations for colonization in vivo.

In summary, we have found that regulation of the ciaB gene in C. jejuni is independent of the alternative sigma factor σ⁵⁴ and that co-transcription of Cia proteins with the flagellar secretory apparatus genes does not seem to occur in C. jejuni. Furthermore, as indicated by the promoter sequences upstream of the ciaB gene, it might be regulated by the rpoD gene. Our results have also shown that the hook filament junction protein, FlgK, is required to complete the flagellar assembly of C. jejuni, and disruption of this gene results in a defective flagellar secretory apparatus and a decrease in the ability to colonize the ceca of birds. The mutations in the rpoN and fliA genes impaired the ability of C. jejuni to colonize the chicken cecum in a more severe manner than comparable flagellar structural mutants involving the filament components FlaA and FlaB. This suggests that both fliA and rpoN can exert indirect regulatory effects involving C. jejuni pathogenesis, in addition to their regulatory control over flagellar class II and III genes. This hypothesis is supported by evidence provided by Goon et al., (2006) that a σ²⁸ controlled non-flagellar gene contributes to virulence of C. jejuni 81-176. We believe that the colonization levels observed in these three defined mutants are due to disruptions in the specific genes and not due to any polar effects of downstream genes, as we have selected for non-polar transformants. Also the observed colonization loads of the mutants in the present trial are based on a statistically sound sample of 20 birds, and are in good agreement with the results of Hendrixson and DiRita (2004) who also found flgk, fliA and rpoN transposon mutants are highly attenuated for cecal colonization.

### Example 6

### Materials and Methods

Bacterial strains and growth conditions. C. jejuni NCTC11168 VI (Carrillo et al., J Biol Chem 279:20327-20338, 2004) was cultured on Mueller Hinton agar (Difco) or Mueller Hinton agar supplemented with 5% bovine citrated blood (PML Microbiologicals, Richmond, BC, Canada) under microaerophilic conditions at 42° C. Escherichia coli DH10B or JM109 were used as hosts for the cloning experiments, and plasmid pPCR-Script Amp (Stratagene, La Jolla, Calif.) was used as a cloning vector. E. coli was grown in Lauria-Bertani (LB) medium (Sigma) at 37° C. The soft agar used for assaying motility of C. jejuni strains consisted of 0.4% (w/v) Bacto agar (Difco) in Mueller Hinton broth (Difco). When appropriate, antibiotics were added to the following final concentrations: 50 μg/ml of kanamycin and 100 μg/ml of ampicillin or carbenicillin. The fliA mutant used in this study was kindly supplied by Christine Szymanaski, Institute for Biological Sciences, NRC, Ottawa, Canada and was generated as described by Carrillo et al (2004).

Construction and characterization of C. jejuni rpoN and flgk mutants. For construction of the rpoN mutant, a wild-type copy of the rpoN gene (Cj0670) was amplified with primers (rpoNF, forward primer, 5′-AAATCACCCAAGCACCTAAGACTAA; and rpoNR, reverse primer, 5′-TAATACGACTCACTATAGGGTTGCTATATTAAGATGTTTGCGATA) from C. jejuni NCTC11168 (CJ1) genomic DNA, and was cloned into pPCR-Script Amp. A kanamycin resistance-cassette was inserted into the unique Swa1 restriction site of rpoN in the vector. For the flgk mutant, a wild-type copy of theflgk gene (Cj 1466) was amplified from CJ1 using the primers (flgkF, forward primer, 5′-TACACAGGTGTTACAGGCTTAAAGG; and flgkR, reverse primer, 5′-TAATACGACTCACTATAGGGAAGCCCTAGTAAGGTATCAAGCATT). The kanamycin-resistance cassette was inserted into the Sty1 site of the flgk gene cloned into pPCR-Script Amp vector. Antibiotic-resistant colonies carrying the kanamycin-resistance cassette in the nonpolar orientation with the cloned gene were identified by restriction analyses of the plasmid DNA and confirmed by DNA sequencing. These kan^{r} modified constructs were electroporated into CJ1 and the kanamycin-resistant transformants were characterized by PCR to confirm that the plasmid had integrated by a double crossover recombination event.

Analysis of C. jejuni protein secretion. C. jejuni were grown on MH agar plates supplemented with 0.1% (w/v) sodium deoxycholate overnight at 37° C. under microaerobic conditions. The bacteria were washed twice in Eagle's minimum essential medium (EMEM) and resuspended to an optical density (OD₆₀₀) of 0.3 (1.0×10³ CFU/ml) in EMEM supplemented with 0.5% fetal bovine serum (FBS). The media were passed through an Amicon ultracentrifugal filter (30K) device (Millipore, USA) to remove albumin. Metabolic labelling experiments were done using 3 ml of EMEM, with and without [³⁵S]-methionine, as described elsewhere (Konkel and Cieplak Infect Immun 60:4945-4949, 1992). After a 3 h metabolic labelling period, bacterial cells were pelleted by centrifugation at 6000 g and the supernatant was filtered through a 0.2 μM filter (Nalgene, USA). Supernatants were concentrated 4-fold by the addition of 5 volumes of ice-cold 1 mM HCl acetone, incubation at 4° C. for 15 min and finally centrifugation at 10,000 g for 20 min. Proteins were resolved by SDS-PAGE with the discontinuous buffer system described by Laemmli (Laemmli, Nature 227:680-685, 1970). The gels were treated with autoradiography enhancer (NEN Life Sciences, USA) according to the supplier's instruction and autoradiography was performed with BioMax MR Film (Kodak, USA) at −80° C.

RNA isolation and reverse transcription-PCR. Overnight cultures of C. jejuni grown on MH agar plates were resuspended to an optical density (OD₆₀₀) of 0.3 in EMEM supplemented with 1% FBS and incubated for 2 hrs at 37° C. under microaerobic conditions. Total RNA was extracted from 1 ml of the culture using the Qiagen RNeasy mini kit (Qiagen) according to the manufacturer's specifications. Purified RNA was treated with RNase-free DNAse 1 (Invitrogen) at 1 unit/1 μg RNA for 30 min at 37° C. followed by addition of 1 μl of 25 mM EDTA to remove contaminant traces of genomic DNA. RNA concentrations were determined by absorbance at 260 nm and RNA integrity was determined using checked by an Agilent 2100.Bioanalyzer. One microgram of DNase I-treated RNA was used for preparation of cDNA with 1 μl of the random hexamer primers (50 ng/μl) provided with the ThermoScript™ RT-PCR kit (Invitrogen) according to manufacturer's instructions. The cDNA was subjected to PCR amplification with primers within the coding region of the ciaB gene (forward primer, 5′-TTGTGAGCGAAGCTATGGTG; reverse primer, 5′-CAGCTTCTTGCCAAGCTTTT). The cycling conditions were as follows: 5 min at 96° C. followed by 29 cycles at 96° C. for 30 s, 50° C. for 40 s and 72° C. for 1 min and a hold at 72° C. for 5 min. Negative controls for the experiment included a no-template control and a no-RT control where RNA was added as a template for the final PCR (to rule out genomic DNA contamination of RNA preparations). The resulting products were resolved by electrophoresis through 1% agarose and bands were visualized using UV light after ethidium bromide staining.

The cell lines used for work on the C. jejuni flagella include INT 407 cells (human intestinal epithelial cells), HeLa cells, and chicken macrophage line HD 11 or chicken fibroblast lines such as CEC-32. The INT 407 cells are used primarily for adherence assays, while the HD11 cells are used for immune assays.

Adherence and invasion assays. The human cervical adenocarcinoma (HeLa) cell line was grown in Eagle's minimum essential medium (EMEM) supplemented with 10% fetal bovine serum (FBS) at 37° C. with 5% CO₂. The adherence and invasion assays were performed as described by Konkel et al. (2000). Briefly, to determine the number of adherent C. jejuni, we inoculated semi-confluent HeLa cell monolayer (10⁵ cells/well) with approximately 1×10⁷ cfu of bacteria and incubated for 3 h in a humidified, 5% CO₂ incubator at 37° C. The infected monolayers were rinsed 3 times with EMEM without FBS, lysed with a solution of 0.1% (vol/vol) Triton X-100 (Calbiochem, USA) and serial dilutions of the lysates were plated on MH-blood plates. The number of viable adherent bacteria was determined by counting the resulting colonies. To measure bacterial internalization, the infected monolayer was washed three times with EMEM and reincubated for another 3 h in fresh EMEM containing 250 μg/ml of gentamicin (Sigma). The number of internalized bacteria was determined as described above. The experiment was conducted with three biological replicates for each cell line and at two time points. Significance of differences among samples was determined with KiruskalWallis one-way ANOVA (Statistix 7, Analytical Software, FL) and a P value of <0.05 was considered significant.

Electron microscopy. C. jejuni, CJ1 as well as rpoN, fliA and flgk mutants were grown on MH agar for 18 h at 42° C. The bacterial cells were resuspended in phosphate-buffered saline and a 200 mesh copper grid coated with Formvar was floated on a drop of this culture for 1 min. The grids were then negatively stained in 0.5% (w/v) sodium phosphotungstate (pH 6), and examined by electron microscopy.

Chick Colonization assays. Colonization of day old chicks by C. jejuni was done as described by Carrillo et al. (2004). Briefly, Leghorn chicks were obtained from a commercial source on the day of hatch. They were randomly allocated into groups of 20 birds and provided with feed and water ad libitum. Birds were cared for in accordance with guidelines of the Canadian Council for Animal Care under procedures approved by the University Committee on Animal Care

Animal Care. In the standard model, five birds in each group were tested for colonization by C. jejuni before infection. Then, all birds were orally infected with the indicated dose of C. jejuni in 0.5 ml of normal saline. To assess the ability of C. jejuni to colonize uninfected birds that were placed in contact with orally challenged birds, 20% of birds were infected and then mingled with the rest of the birds in the group. The infected birds were marked so they could be readily identified. Colonization of the birds was monitored by culturing cloacal swabs on Karmali agar (Bacto, USA). Birds were maintained for 7 days after challenge and then were euthanized by cervical dislocation. Ceca were aseptically collected to determine numbers of C. jejuni present.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

### REFERENCES

Alm, R. A., Guerry, P. & Trust, T., J., (1993) The Campylobacter sigma 54 flaB flagellin promoter is subject to environmental regulation. J Bacteriol 175, 4448-4455.
Debabarata Biswas, Fernando Ursla, Reiman Carla, Phillip Wilson, Andrew Potter and Brenda Allan (2006) Correlation between in vitro secretion of virulence-associated proteins of Campylobacter jejun and colonization of chickens. (In press) Current Microbiology
Carrillo, C. D., Taboada, E., Nash, J. H. & 15 others (2004) Genome-wide expression analyses of Campylobacter jejuni NCTC11168 reveals coordinate regulation of motility and virulence by flhA. J Biol Chem 279, 20327-20338; Golden and Acheson, Infect Immun 70:1761-1771, 2002. Identification of motility and autoagglutination Campylobacter jejuni mutants by random transposon mutagenesis. Infect Immun 70, 1761-1771; Goon, S., Ewing, C. P., Lorenzo, M., Pattarini, D., Majam, G. & Guerry, P. (2006) A σ²⁸-regulated nonflagella gene contributes to virulence of Campylobacter jejuni 81-176. Infect Immun 74, 769-772.
Grant, C. C. R., Konkel, M. E., Cieplak, W., Jr.& Tompkins, L. S. (1993) Role of flagellar in adherence, internalization and translocation of Campylobacter jejuni in nonpolarized and polarized epithelial cell cultures. Infect Immun 61, 1764-1771
Hendrixson, D. R., Akerley, B. J. & DiRita, V. J. (2001) Transposon mutagenesis of Campylobacter jejuni identifies a bipartite energy taxis system required for motility. Mol Microbiol 40, 214-224
Hendrixson, D. R. & DiRita, V. J. (2003) Transcription of σ⁵⁴-dependent but not σ²⁸-dependent flagellar genes in Campylobacter jejuni is associated with formation of the flagellar secretory apparatus. Mol Microbiol 50, 687-702
Hendrixson, D. R. & DiRita, V. J. (2004) Identification of Campylobacter jejuni genes involved in commensal colonization of the chick gastrointestinal tract. Mol Microbiol 52, 471-484.
Jagannathan, A., Constantinidou, C. & Penn, C. W. (2001) Roles of rpoN, fliA, and flgR in expression of flagella in Campylobacter jejuni. J Bacteriol 183, 2937-2942
Kazmierczak, M. J., Wiedmann, M. & Boor, K. J. (2005) Alternative sigma factors and their roles in Bacterial virulence. Microbiol. & Mol Biology Reviews 69, 527-543
Konkel, M. E., & Cieplak, W. (1992) Altered synthetic response of Campylobacter jejuni to co-cultivation with human epithelial cells is associated with enhanced internalization. Infect Immun 60, 4945-4949.
Konkel, M. E., Corwin, M. D., Joens, L. A., & Cieplak, W. (1992) Factors that influence the interaction of Campylobacter jejuni with cultured mammalian cells. J Med Microbiol 37, 30-37.
Konkel et al., Mol Microbiol 32:691-701, 1999) Bacterial secreted proteins are required for the internalization of Campylobacter jejuni into cultured mammalian cells. Mol Microbiol 32, 691-701
Konkel, M. E., Klena, J. D., Rivera-Amill, V., Monteville, M. R., Biswas, D., Raphael, B. & Mickelson, J. (2004) Secretion of virulaence proteins from Campylobacter jejuni is dependent on a functional flagellar export apparatus. J Bacteriol 186, 3296-3303
Kumar, A., Malloch, R. A., Fujita, N., Smillie, D. A., Ishihama, A. & Hayward, R. S. (1993) The minus 35-recognition region of Escherichia coli sigma 70 is inessential for initiation of transcription at an extended minus 10 promoter. J Mol Biol 232, 406-418
Laemmli, U. K. (1970) Cleavage of structural proteins during the assembly of the head of bacteriophage T4. Nature 227, 680-685
Muramoto, K., Makishima, S., Aizawa, S., & MacNab, R. (1999) Effect of hook subunit concentration on assembly and control of length of the flagellar hook of Salmonella J Bacteriol 181, 5808-5813
Newell, D. G., & Feamley, C. (2003) Sources of Campylobacter colonization in broiler chickens. Appl Environ. Microbiol. 69, 4343-4351.
Rivera-Amill, V. & Konkel, M. E. (1999) Secretion of Campylobacter jejuni Cia proteins is contact dependent. In Mechanisms in the pathogenesis of enteric diseases, pp 225-229. Edited by P S Paul and D H Francis, New York: Plenum Publishing Corp.
Young, C. R., Ziprin, R. L., Hume, M. E. & Stanker, L. H. (1999) Dose response and organ invasion of day-of-hatch Leghorn chicks by different isolates of Campylobacter jejuni. Avian Dis 43, 763-767.
Wassenaar, T. M., Bleumink-Pluym, N. M. & van der Zeijst, B. A. (1991) Inactivation of Campylobacter jejuni flagellin genes by homologous recombination demonstrates that flaA but not flaB is required for invasion. EMBO J. 10, 2055-2061.
Ziprin, R. L., Young, C. R., Byrd, J. A., Stanker, L. H., Hume, M. E., Gray, S. A., Kim, B. J. & Konkel, M. E. (2001) Role of Campylobacter jejuni potential virulence genes in cecal colonization. Avian Diseases 45, 549-557.
Ziprin, R. L., Harvey, R. B., Hume, M. E. & Kubena, L. E. (2002) Campylobacter colonization of the crops of newly hatched leghorn chicks. Avian Diseases 46, 985-988.

What is claimed:

1. A vaccine composition comprising an effective immunizing amount of a bacterial flagellar protein or a protein involved in bacterial flagellar biosynthesis and a pharmaceutically acceptable carrier, wherein said vaccine composition is effective in a vertebrate subject to reduce or eliminate Gram-negative bacterial infection.

2. The vaccine of claim 1 wherein the bacterial flagellar protein is a bacterial FlgK protein.

3. The vaccine of claim 1 wherein the protein involved in bacterial flagellar biosynthesis is a bacterial RpoN protein or a bacterial FliA protein.

4. The vaccine of claim 1 wherein the protein involved in bacterial flagellar biosynthesis is regulated by a bacterial RpoN protein.

5. The vaccine of claim 1 wherein the protein involved in bacterial flagellar biosynthesis is regulated by a bacterial FliA protein.

6. The vaccine of claim 1 wherein the infection is Campylobacter jejuni infection.

7. The vaccine of claim 1 wherein the vertebrate subject is a mammalian subject or an avian subject.

8. The vaccine of claim 6 wherein the avian subject is one day of age or older.

9. The vaccine of claim 6 wherein the avian subject is in ovo.

10. The vaccine of claim 1 further comprising an immunological adjuvant.

11. The vaccine of claim 10, wherein the immunological adjuvant comprises an oil-in-water emulsion.

12. The vaccine of claim 11, wherein the immunological adjuvant comprises a mineral oil and dimethyldioctadecylammonium bromide.

13. The vaccine composition of claim 12, wherein the immunological adjuvant is VSA3.

14. The vaccine composition of claim 13, wherein VSA3 is present in the composition at a concentration of about 20% to about 40% (v/v).

15. The vaccine composition of claim 14, wherein VSA3 is present in the composition at a concentration of about 30% (v/v).

16. The vaccine composition of claim 1, further comprising one or more recombinant or purified antigens selected from the group consisting of FlgK protein, RpoN protein, or FliA protein.

17. The vaccine composition of claim 1, further comprising one or more recombinant or purified antigens selected from the group consisting of a bacterial protein regulated by RpoN protein, or a bacterial protein regulated by FliA protein.

18. The vaccine composition of claim 17, wherein one or more of FlgK protein, RpoN protein, or FliA protein comprise at least 20% of the cell protein present in the composition.

19. A method for preventing or treating a Gram-negative bacterial infection in a vertebrate subject comprising,
administering a bacterial flagellar protein or a protein involved in bacterial flagellar biosynthesis to the vertebrate subject in an amount effective to reduce or eliminate the bacterial infection.

20. The method of claim 19 wherein the bacterial flagellar protein is a bacterial FlgK protein.

21. The method of claim 19 wherein the protein involved in bacterial flagellar biosynthesis is a bacterial RpoN protein or a bacterial FliA protein.

22. The method of claim 19 wherein the protein involved in bacterial flagellar biosynthesis is regulated by a bacterial RpoN protein.

23. The method of claim 19 wherein the protein involved in bacterial flagellar biosynthesis is regulated by a bacterial FliA protein.

24. The method of claim 19 wherein the infection is Campylobacter jejuni infection.

25. The method of claim 19 wherein the vertebrate subject is a mammalian subject or an avian subject.

26. The method of claim 25 wherein the avian subject is one day of age or older.

27. The method of claim 25 wherein the avian subject is in ovo.

28. The method of claim 19, wherein the immunological adjuvant comprises an oil-in-water emulsion.

29. The method of claim 28, wherein the immunological adjuvant comprises a mineral oil and dimethyldioctadecylammonium bromide.

30. The method of claim 29, wherein the immunological adjuvant is VSA3.

31. The method of claim 30, wherein VSA3 is present in the composition at a concentration of about 20% to about 40% (v/v).

32. The method of claim 31, wherein VSA3 is present in the composition at a concentration of about 30% (v/v).

33. The method of claim 19, further comprising one or more recombinant or purified antigens selected from the group consisting of FlgK protein, RpoN protein, or FliA protein.

34. The method of claim 19, further comprising one or more recombinant or purified antigens selected from the group consisting of a bacterial protein regulated by RpoN protein, or a bacterial protein regulated by FliA protein.

35. The method of claim 34, wherein one or more of FlgK protein, RpoN protein, or FliA protein comprise at least 20% of the cell protein present in the composition.

36. A method for eliciting an immunological response in a vertebrate subject against a Gram-negative bacterial infection comprising,
administering a bacterial flagellar protein or a protein involved in bacterial flagellar biosynthesis to the vertebrate subject in an amount effective to reduce or eliminate the bacterial infection.

37. The method of claim 36 wherein the bacterial flagellar protein is a bacterial FlgK protein.

38. The method of claim 36 wherein the protein involved in bacterial flagellar biosynthesis is a bacterial RpoN protein or a bacterial FliA protein.

39. The method of claim 36 wherein the protein involved in bacterial flagellar biosynthesis is regulated by a bacterial RpoN protein.

40. The method of claim 36 wherein the protein involved in bacterial flagellar biosynthesis is regulated by a bacterial FliA protein.

41. The method of claim 36 wherein the infection is Campylobacter jejuni infection.

42. The method of claim 36 wherein the vertebrate subject is a mammalian subject or an avian subject.

43. The method of claim 42 wherein the avian subject is one day of age or older.

44. The method of claim 42 wherein the avian subject is in ovo.

45. A method for reducing colonization of Gram negative bacteria in an avian species comprising administering to the avian species a composition comprising a bacterial flagellar protein or a protein involved in bacterial flagellar biosynthesis and an immunological adjuvant in an amount effective to reduce Gram negative bacterial count in the avian species.

46. The method of claim 45 wherein reducing colonization of Gram negative bacteria in the avian species further comprises reducing a risk of infectious transfer from the avian species to humans.

47. The method of claim 45 wherein the bacterial flagellar protein is a bacterial FlgK protein.

48. The method of claim 45 wherein the protein involved in bacterial flagellar biosynthesis is a bacterial RpoN protein or a bacterial FliA protein.

49. The method of claim 45 wherein the protein involved in bacterial flagellar biosynthesis is regulated by a bacterial RpoN protein.

50. The method of claim 45 wherein the protein involved in bacterial flagellar biosynthesis is regulated by a bacterial FliA protein.
